(19) United States
(12) Patent Application Publication
(10) Pub. No.: US 2023/0213863 A1
(43) Pub. Date: Jul. 6, 2023

(54) COMPOUND, RESIST UNDERLAYER FILM COMPOSITION INCLUDING SAME, AND RESIST UNDERLAYER FILM

(71) Applicant: DONGJIN SEMICHEM CO., LTD., Incheon (KR)

(72) Inventors: Young Bae LIM, Hwaseong-si (KR); Hyun Jin KIM, Hwaseong-si (KR); Jong Won KIM, Hwaseong-si (KR); Ji Hyun KIM, Hwaseong-si (KR); Bo Lyong KIM, Hwaseong-si (KR); Kun Hee KIM, Hwaseong-si (KR); Eun Jeong CHO, Hwaseong-si (KR); Seung Mook LEE, Hwaseong-si (KR); Sang Hoon CHANG, Hwaseong-si (KR)

(21) Appl. No.: 18/090,313

(22) Filed: Dec. 28, 2022

(30) Foreign Application Priority Data
Dec. 30, 2021 (KR) 10-2021-0193544

(51) Int. Cl. G03F 7/09; C08F 112/32

(52) U.S. Cl. CPC G03F 7/092; C08F 112/32

(57) ABSTRACT

A compound with enhanced etching resistance, gap-filling properties, and heat resistance includes a repeating unit represented by Formula 1.

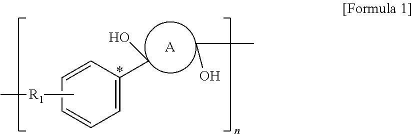

### CROSS REFERENCE TO RELATED APPLICATION

The present application claims priority to Korean Patent Application No. 10-2021-0193544, filed Dec. 30, 2021, which is incorporated herein by reference in its entirety.

### BACKGROUND

### 1. Field of the Disclosure

The present disclosure relates to a compound, a resist underlayer film composition including the same, and a resist underlayer film. More specifically, the present disclosure relates to a poly compound with enhanced gap-filling characteristics, etching resistance, and heat resistance. The present disclosure also relates to a resist underlayer film composition including the same, and a resist underlayer film.

### 2. Description of the Related Art

Recently, with growing demand for more highly integrated and faster large-scale integrated circuits (LSIs), semiconductor patterns have become scaled down. In addition, lithography techniques using light exposure, currently used as general-purpose technology, are approaching the inherent resolution limit imposed by the wavelength of light.

As pattern sizes decrease with the miniaturization and increasing density of semiconductor devices, the thickness of photoresist films and patterns has gradually decreased to prevent the collapse of photoresist patterns. However, it is more difficult to etch a target layer when the use of a photoresist pattern with a small thickness. Therefore, a technique of interposing an organic or inorganic film with good etching resistance between a photoresist film and a target layer to be etched has been used. Such an interposed organic or inorganic film is termed as a “resist underlayer film”′, and a process in which the resist underlayer film is etched to obtain an underlayer film pattern, and the etching target layer is etched with the use of the underlayer film pattern is termed as a “resist underlayer film patterning process”.

Typically, the inorganic underlayer film used in the resist underlayer film patterning process is made from silicon nitride, silicon oxynitride, polysilicon, titanium nitride, amorphous carbon, or the like by chemical vapor deposition (CVD). The underlayer film formed by CVD has good etching selectivity and etching resistance but also has various problems such as the generation of particles, low productivity, high costs, and the like. To solve such problems, active research has been conducted on spin-coating organic underlayer films instead of CVD underlayer films.

Compounds used to form typical organic underlayer films have an advantage of high-carbon structures. However, due to the disadvantage of low solubility, it is difficult to control the thickness of a resist underlayer film when the resist underlayer is made of an existing compound. In addition, due to the low degree of cross-linking, it is difficult to form a resist underlayer film having a uniform thickness in a process involving heat. As a result, there is a problem in that the performance of an underlayer film as a hardmask is deteriorated.

### SUMMARY

An objective of the present disclosure is to provide a compound that forms a resist underlayer film with an increased density leading to enhanced etching resistance in a low-temperature or high-temperature process and which effectively prevents the bending of a pattern during an etching process.

Another objective of the present disclosure is to provide a compound that has good gap-filling characteristics to prevent the occurrence of voids when a resist underlayer film composition is used to coat a substrate, thereby enabling the implementation of a planar underlayer film.

A further objective of the present disclosure is to provide a resist underlayer film composition including the compound.

Another objective of the present disclosure is to provide a resist underlayer film as a cured product of the resist underlayer film composition.

Still another objective of the present disclosure is to provide a method of manufacturing a resist underlayer film that is easily adjustable in thickness thereof.

Objectives of the present disclosure are not limited to the objectives mentioned above. Other objectives and advantages of the present disclosure not mentioned will be understood by the following description, and will be more apparent from the embodiments of the present disclosure. In addition, the objectives and advantages of the present disclosure will be realized by the means of the appended claims, and combinations thereof.

An embodiment of the present disclosure to achieve the above objectives provides a compound containing a repeating unit represented by Formula 1.

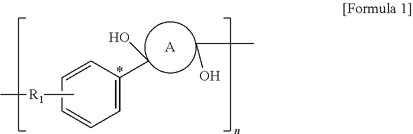

In Formula 1, R₁ has 1 to 5 substituents that are present in a carbon (*)-containing benzene ring, A is a substituted or unsubstituted monocyclic ring or polycyclic ring, and n is the number of the repeating units in a range of 1 to 55.

Another embodiment of the present disclosure to achieve the above objectives provides a resist underlayer film composition including the compound and a solvent.

Yet another embodiment of the present disclosure to achieve the above objectives provides a resist underlayer film as being a cured product of the resist underlayer film composition.

Means to solve the above problems do not list all the features of the present disclosure. Various features of the present disclosure and its advantages and effects will be understood in more detail with reference to the following specific embodiments.

According to an aspect of the present disclosure, the density of a resist underlayer film in a low-temperature or high-temperature process may increase, so that the etching resistance of the resist underlayer film is enhanced and the bending of the pattern can be effectively prevented during an etching process. Furthermore, according to another aspect of the present disclosure, when the resist underlayer film composition is used to coat a substrate, voids may not be generated, and a planar underlayer film can be obtained due to good gap-filling characteristics of the film composition.

The above-described effects and specified effects of the present disclosure will be also described in the following description for carrying out the present disclosure.

### BRIEF DESCRIPTION OF THE DRAWINGS

FIG. 1 shows field emission scanning electron microscope (FE-SEM) images of a resist underlayer film prepared according to Example 1;

FIG. 2 shows FE-SEM images of a resist underlayer prepared according to Comparative Example 1;

FIG. 3 shows FE-SEM images of a resist underlayer prepared according to Comparative Example 2;

FIG. 4 shows FE-SEM images of a resist underlayer prepared according to Comparative Example 3;

FIG. 5 shows FE-SEM images of a resist underlayer prepared according to Reference Example 2; and

FIG. 6 shows FE-SEM images of a resist underlayer prepared according to Reference Example 2.

### DETAILED DESCRIPTION

Hereinbelow, each configuration of the present disclosure will be described in detail such that the disclosure can be easily embodied by those skilled in the art. However, the configurations are merely examples and the scope of the present disclosure is not limited to the following description.

Herein, “substituted” means that any one hydrogen atom is replaced with any one selected from the group consisting of a halogen atom, a hydroxyl group, a carboxyl group, a nitro group, an amine group, a thio group, a thiol group, an alkoxy group, a nitrile group, an aldehyde group, an ether group, an ester group, an acetal group, a ketone group, an alkyl group, a cycloalkyl group, a heterocycloalkyl group, an allyl group, a benzyl group, an aryl group, a heteroaryl group, derivatives thereof, and combinations thereof.

In the present disclosure, one embodiment provides a compound containing a repeating unit represented by Formula 1.

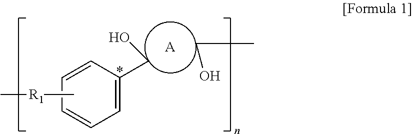

Hereinafter, the configurations of the present disclosure will be described in further detail.

### 1. Compound

An embodiment of the present disclosure provides the compound containing the repeating unit represented by Formula 1.

Herein, 1 to 5 substituents in a carbon (*)-containing benzene ring may be defined as encompassing all of: a substituent that is present at a position selected from ortho position, meta position, para position, and combinations thereof around the carbon (*); or a substituent forming a fused ring with the carbon (*)-containing benzene ring, which is the parent body. For example, to form the fused ring, R₁ may be defined as having 2 to 5 substituents and each substituent may be bonded to each other to form a monocyclic ring or a polycyclic ring. The monocyclic ring or polycyclic ring may be, for example, an aliphatic ring or aromatic ring. As a further specific example, a repeating unit having 2 substituents that is present in the carbon (*)-containing benzene ring may include a repeating unit represented by Formula 1l and a repeating unit represented by Formula 1a. The repeating unit represented by Formula 1l may mean that substituents are each independently para-substituted and meta-substituted around the carbon (*). In addition, the repeating unit represented by Formula 1a may mean that substituents, which are each independently para-substituted and meta-substituted around the carbon (*), are bonded to each other to form the fused ring.

In Formula 1, R₁ has 1 to 5 substituents that are present in the carbon (*)-containing benzene ring, A is a substituted or unsubstituted monocyclic ring or polycyclic ring, and n is the number of the repeating units in a range of 1 to 55. Specifically, n is a number in a range of 2 to 15. In formula 1, when n falls within the above numerical range, a resist underlayer film with enhanced etching resistance, gap-filling characteristics, and heat resistance may be provided.

According to an embodiment of the present disclosure, the substituted monocyclic ring or polycyclic ring may contain a polar functional group. The polar functional group may include at least one selected from the group consisting of a hydroxyl group, a thiol group, and carboxyl group. Specifically, the polar functional group may include a hydroxyl group.

According to another embodiment of the present disclosure, the substituted monocyclic ring or polycyclic ring may not include a polar functional group. That is, when a structure A in Formula 1 does not contain a polar functional group, etching resistance may be further enhanced. In addition, an aspect ratio of the resist underlayer film formed on a part having a large pattern thickness (a line part) and a part having a small pattern thickness (a space part) may be further decreased.

Herein, 2 to 3 substituents which is meta-substituted or para-substituted around the carbon (*) may be defined as encompassing a substituent forming a fused ring with the carbon (*)-containing benzene ring, which is the parent body. For example, to form the fused ring, R₁ may be defined as having 2 or 3 substituents and each substituent may be bonded to each other to form a monocyclic ring or polycyclic ring. The monocyclic ring or polycyclic ring may be an aliphatic ring or aromatic ring.

R₁ may have 2 or 3 substituents which are meta-substituted and para-substituted around the carbon (*), and the 2 or 3 substituents which are meta-substituted and para-substituted may be bonded to each other to form a monocyclic ring or polycyclic ring. Specifically, R₁ has a molecular structure represented by Formula 2

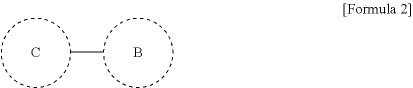

In Formula 2, B may be bonded to the carbon (*)-containing benzene ring in Formula 1 and may contain a molecular structure of a monocyclic aromatic hydrocarbon or polycyclic aromatic hydrocarbon which is substituted with a polar functional group. C may contain a molecular structure of a monocyclic aromatic hydrocarbon or polycyclic aromatic hydrocarbon which is substituted with a polar functional group. Both B and C contain the molecular structure of a monocyclic aromatic hydrocarbon or polycyclic aromatic hydrocarbon with a high carbon content. As a result, etching resistance may be enhanced, and a vertical pattern profile may be thus easily formed. In addition, solubility in a solvent may increase due to the polar functional group, so that a thickness may be more easily adjustable. Furthermore, the number of positions capable of being cross-linked may increase in the entire molecular structures, thereby implementing a uniform film.

The polar functional groups of B and C may each independently include at least one selected from the group consisting of a hydroxyl group, an amine group, a thiol group, and a carboxyl group. An existing amorphous carbon layer (ACL) contains a high content of carbon, and thus has an advantage of good etching resistance. However, there have been problems with voids and planarization characteristics when filling a gap of a fine underlying film layer. In addition, since a film layer is formed by chemical vapor deposition, there are various problems such as the generation of particles, low productivity, high costs, and the like. According to an embodiment of the present disclosure, when Formula 2 contains the polar functional group, solubility in a solvent may increase, so the thickness of the resist underlayer film may be more easily adjustable. In addition, a thin film may be laminated by spin coating, so there is an effect of reducing process costs. Furthermore, as various polymer structures may be controlled, process-friendly effects may be realized by controlling hydrophilicity, absorbance, and refractive index. Moreover, the etching resistance, which is relatively insufficient compared to that of the amorphous carbon layer, may be complemented by increasing a thickness of the film layer, and the problem with voids may be solved by adjusting the polymer structure and molecular weight when filling the gap of the fine underlying film layer. As a result, good planarization characteristics can be obtained. Particularly, when the polar functional group includes a hydroxyl group, the resist underlayer film having a large thickness may be easily implemented. In addition, the aspect ratio of the resist underlayer film formed on the part having a large pattern thickness (the line part) and the part having a small pattern thickness (the line part) can be further decreased.

Monomers configured to form the structure of B and C may be, for example, compounds represented by Formulas B-1 to B-25. The compounds may each independently contain at least two benzene rings and symmetrically contain a halide atom. As the monomers configured to form the structure of B and C symmetrically contain the halide atom, cross-linking sites in polymer chains may be extended in longitudinal and lateral directions during a polymerization reaction. As a result, the resist underlayer film may be formed in a packing structure, which increases the degree of uniformity in the film, thereby easily forming patterns without bending during an etching process.

As another example, a monomer configured to form the structure of B and C may be a compound containing at least two benzene rings and symmetrically containing a hydroxyl group. As the monomer configured to form the structure of B and C symmetrically contains the halide atom, cross-linking sites in polymer chains may be extended in longitudinal and lateral directions during a thermal cross-linking reaction. As a result, the resist underlayer film may be formed in a packing structure, which increases the degree of uniformity in the film, thereby easily forming patterns without bending during the etching process.

According to another embodiment of the present disclosure, the compound may include first and second molecular structures at first and second terminal ends, respectively. The first and second molecular structures may be the same or different, and preferably, are the same. Particularly, when the first and second molecular structures are the same, uniformity of etching, which means the degree of uniformity in an etched part, may further increase.

The first and second molecular structure may be molecular structures derived from compounds capable of terminating polymer chain extension.

According to another embodiment of the present disclosure, the first and second molecular structures may be each independently: a molecular structure derived from a ketone-based compound; or a molecular structure derived from an aryl halide-based compound. The first and second molecular structures may contain a polar functional group. The polar functional group may include at least one selected from the group consisting of a hydroxyl group, an amine group, a thiol group, and a carboxyl group. According to an embodiment of the present disclosure, as the compounds contains not only the repeating unit but also the polar functional groups at the terminal ends of the polymer chain, solubility of the compound in the solvent may be further increased. As a result, the thickness of the resist underlayer film may be easily adjustable.

Specifically, the ketone-based compound may be a fluorenone-based compound, a cyclohexanone-based compound, a cyclopentanone-based compound, and the like. Specifically, the ketone-based compound may be a fluorenone-based compound. The fluorenone-based compound may be, for example, fluorenone, fluorenone containing a hydroxyl group, fluorenone containing a carboxyl group, and the like. The ketone-based compound may form a molecular structure containing a hydroxyl group by being provided with electrons from a nucleophile produced by a reaction between the monomers and an organometallic reagent. In addition, the ketone-based compound may terminate the polymer chain extension.

The aryl halide-based compound may be, for example, a compound containing a single halide atom, unlike the compounds represented by Formulas B-1 to B-25, in which 2 halide atoms are symmetrically contained. However, the scope and spirit of the present disclosure are not limited thereto, and any aryl halide-based compounds may be applied.

According to yet another embodiment of the present disclosure, a ratio of a total number of polar functional groups to a total number of rings (the total number of the polar functional groups: the total number of the rings) in the compound may be in a range of 1:0.5 to 1:6, specifically, is in the range of 1:1 to 1:4, and more specifically, is in the range of 1:1.2 to 1:3.2. When the ratio of the total number of polar functional groups to the total number of rings falls within the above ranges, the aspect ratio of the resist underlayer film formed on the part having a large pattern thickness (the line part) and the part having a small pattern thickness (the space part) may be further decreased, and thus aspect ratio performance may be significantly enhanced. In addition, the resist underlayer film with a high degree of cross-linking even at low curing temperatures may be achieved, and good etching resistance and heat resistance may be implemented. Furthermore, as the thickness of the resist underlayer film is uniformly formed, the bending of the pattern may be effectively prevented during the etching process, and the vertical pattern profile may be thus easily formed.

In the related art, there has been an attempt to construct a resist underlayer film composition using a compound to which a polar functional group is applied. However, even though the polar functional group is applied to the compound, when the content of carbon is low, etching resistance decreases. As a result, there is a problem in that the vertical pattern profile is difficult to form. In addition, when a size of a monomer is bulky, there is a problem in that gap-filling characteristics are deteriorated and thus, voids may be generated. According to an embodiment of the present disclosure, the compound capable of obtaining both etching resistance and gap-filling characteristics may be provided by deriving the optimal range of the ratio of the total number of the polar functional groups to the total number of the rings (the total number of the polar functional groups: the total number of the rings).

The compound, according to the present disclosure, may have a weight average molecular weight (Mw) in a range of 800 g/mol to 20,000 g/mol, specifically, in the range of 1,000 g/mol to 10,000, in the range of 1,000 g/mol to 6,000 g/mol, or in the range of 1,500 g/mol to 6,000 g/mol. When the weight average molecular weight of the compound falls within the above numerical range, etching resistance may be enhanced due to an increase in the density of the resist underlayer film. In addition, the bending of the pattern may be effectively prevented during the etching process. Furthermore, when the resist underlayer film composition is used to coat a substrate, the gap-filing characteristics are good. As a result, the occurrence of voids may be prevented and the planar underlayer film can be obtained.

The repeating unit represented by Formula 1 may be, for example, any one compound selected from the group consisting of compounds represented by Formulas 1a to 1l. In Formulas 1a to 1l, n is the number of the repeating units, and may be in a range of 1 to 55. However, the scope and spirit of the present disclosure are not limited thereto, and the repeating unit may be a repeating unit composed of combinations of various monomers according to the following Synthesis Preparation Examples to be described.

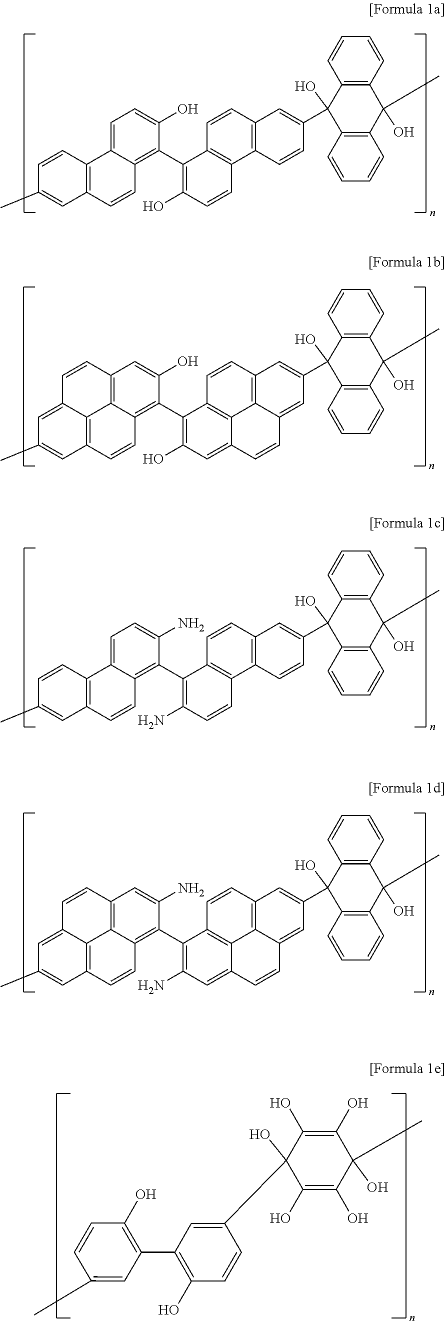

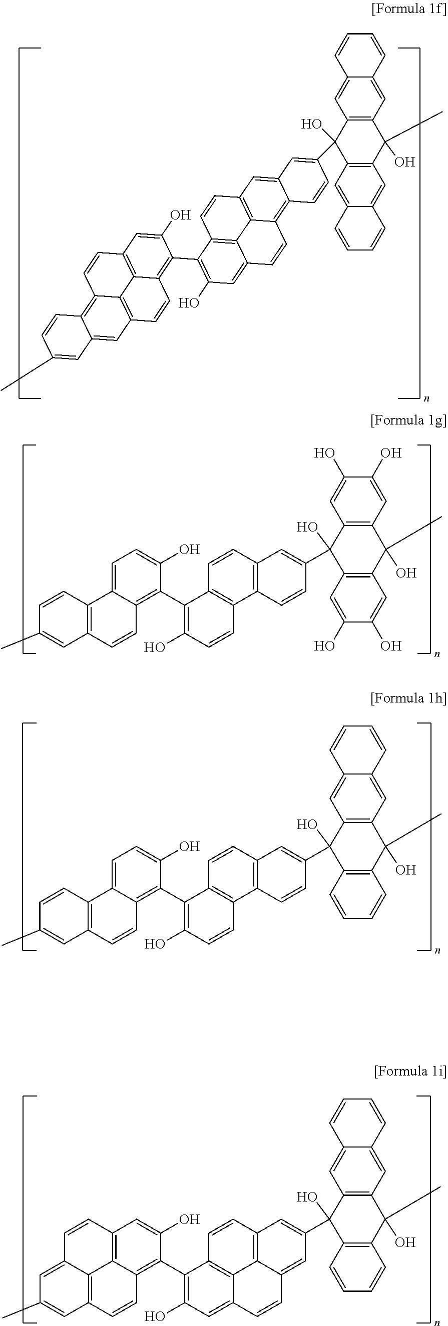

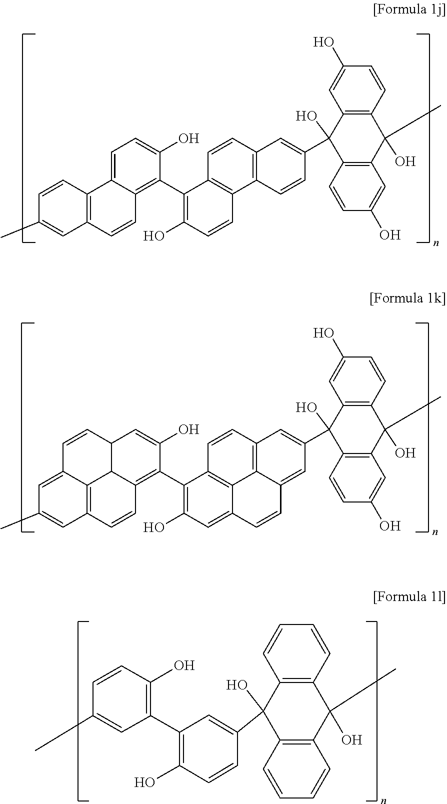

### 2. Resist Underlayer Film Composition

Another embodiment of the present disclosure, may provide a resist underlayer film composition including the compound and a solvent. The features already described and repeated descriptions will be briefly described or omitted.

According to the present disclosure, the compound contained in the resist underlayer film composition may account 2.0% to 30.0% by weight, specifically, 2.0% to 25.0% by weight, and more specifically, 2.0% to 20.0% by weight, with respect to a total weight of the resist underlayer film composition. When the content of the compound is less than the above numerical range, the resist underlayer film may not be formed. In addition, when the content of the compound exceeds the above numerical range, there is a concern of deterioration in the film quality of the resist underlayer during coating with the resist underlayer film.

The solvent, according to the present disclosure, is a solvent having a solubility in the compound, and may be appropriately selected according to the type of the compound. The solvent may be, for example, a solvent selected from the group consisting of a ketone-based solvent, an ester-based solvent, an alcohol-based solvent, and an aromatic hydrocarbon solvent, or a solvent mixture of at least two selected from the group consisting of a ketone-based solvent, an ester-based solvent, an alcohol-based solvent, and an aromatic hydrocarbon solvent. Specifically, the solvent may be a solvent selected from the group consisting of cyclohexanone, propylene glycol monomethyl ether, propylene glycol monomethyl ether acetate, ethyl lactate, hydroxyisobutyric acid methyl ester, 1-methoxy-2-propanol, and anisole, or a solvent mixture of at least two selected from the group consisting of cyclohexanone, propylene glycol monomethyl ether, propylene glycol monomethyl ether acetate, ethyl lactate, hydroxyisobutyric acid methyl ester, 1-methoxy-2-propanol, and anisole.

The resist underlayer film composition, according to the present disclosure may further include an additive, as needed. The additive may be, for example, any one selected from the group consisting of a cross-linking agent, an acid catalyst, an acid generator, a surfactant, and combinations thereof. The content of the additive may be appropriately adjusted according to the purpose of use.

The cross-linking agent is added to induce a cross-linking reaction so that the resist underlayer film may be further cured. The cross-linking agent is not particularly limited and may be variously applied. For example, the cross-linking agent included in the compound may be 1 part to 20 parts by weight with respect to 100 parts by weight of the compound. When the content of the cross-linking agent falls within the above numerical range, the cross-linking reaction between polymers may be induced, thereby effectively controlling the degree of curing of the resist underlayer film.

The cross-linking agent, for example, may be selected as any one from the group consisting of a methylol group, a melamine-based cross-linking agent including a cross-link forming substituent, such as methoxymethyl group, an epoxy-based cross-linking agent, and combinations thereof. Typically, MX-270, MX-279, MX-280, MW-390, and the like, which are manufactured by Sanwa Chemical, may be used.

The acid catalyst is a catalyst added to accelerate the cross-linking reaction. For example, the acid catalyst may be an acid compound such as p-toluenesulfonic acid, trifluoromethanesulfonic acid, salicylic acid, citric acid, or the like. The content of the acid catalyst may be appropriately adjusted according to the content of the cross-linking agent.

The acid generator is added to match with the acidity of a resist film, and may be, for example, trifluoromethanesulfonate of bis(4-tert-butylphenyl)iodonium.

The surfactant may be added to enhance coating defects occurring as the content of a non-volatile phase increases when the resist underlayer film is formed. As the surfactant, for example, sulfinol series manufactured by Air Products, F-series (F-410, F-444, F-477, R-08, R-30, etc.) manufactured by DIC, or the like may be used.

### 3. Resist Underlayer Film and Method of Manufacturing Same

Another embodiment of the present disclosure may provide a resist underlayer film including a cured product of the resist underlayer film composition.

The resist underlayer film, according to the present disclosure, has a thickness which can be appropriately adjusted according to the content of the compound. Specifically, the resist underlayer film may have a thickness in a range of 0.03 μm to 1.50 μm, in the range of 0.03 μm to 1.20, or in the range of 0.03 μm to 0.06 μm.

When the content of a polar functional group of a compound applied to an existing resist underlayer film composition is low, there is a problem in that a resist cured film having a large thickness is difficult to be implemented since the solubility of the compound in a solvent is low. However, when applying the compound, according to an embodiment of the present disclosure, to the resist underlayer film composition, the resist underlayer film with a high degree of cross-linking even at low curing temperatures and good etching resistance and heat resistance may be implemented. Furthermore, as the thickness of the resist underlayer film is uniformly formed, the bending of a pattern can be effectively prevented during an etching process, and a vertical pattern profile may be thus easily formed.

Yet another embodiment of the present disclosure may provide a method of manufacturing a resist underlayer film. The method includes coating a substrate with the resist underlayer film composition and curing the resist underlayer film composition.

The step of coating the resist underlayer film on a substrate may be performed by spin coating, roller coating, spraying, or combinations thereof.

The step of curing the resist underlayer film may be performed by heating the substrate coated with the resist underlayer film composition with a heating device at a temperature in a range of 160° C. to 550° C. Specifically, the heating may be performed at a temperature in the range of 240° C. to 400° C. When the range of heating temperature is lower than the above numerical range, not only the solvent contained in the resist underlayer film composition may be insufficiently removed, but also the cross-linking reaction may insufficiently occur. In addition, when the range of heating temperature is higher than the above numerical range, there may be a concern about the resist underlayer film becoming chemically unstable. The heating device is a device capable of heating the resist underlayer film composition and may be, for example, a device such as a hot plate, a convection oven, or the like.

The substrate, according to the present disclosure, may be a semiconductor substrate, for example, a silicon wafer, a germanium substrate, a silicon-germanium substrate, a silicon-on-insulator (SOI) substrate, a germanium-on-insulator (GOI), or the like. The substrate may have a thickness that can be appropriately selected according to the purpose of use.

Hereinafter, the embodiments of the present disclosure will be described in detail so that the present disclosure can be easily embodied by those skilled in the art may, but the embodiments of the present disclosure are disclosed only for illustrative purposes. Therefore, the scope and spirit of the present disclosure are not limited to the embodiment described hereinbelow.

### Synthetic Preparation Example 1: Preparation of Monomers 4-1 to 4-63

TABLE 1

| Name | Formula |
|---|---|
| A-1 | 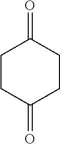 |
| A-2 | 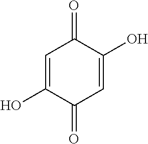 |
| A-3 | 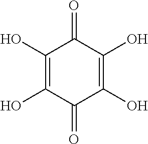 [SMILES: O=C1C(O)=C(O)C(=O)C(O)=C1O] |
| A-4 | 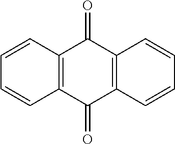 [SMILES: O=C1c2ccccc2C(=O)c2ccccc21] |
| A-5 | 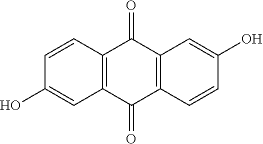 [SMILES: O=C1c2ccc(O)cc2C(=O)c2ccc(O)cc21] |
| A-6 | 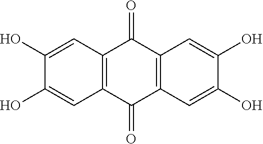 [SMILES: O=C1c2cc(O)c(O)cc2C(=O)c2cc(O)c(O)cc21] |
| A-7 | 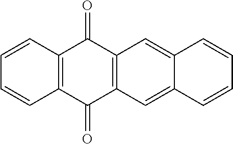 [SMILES: O=C1c2ccccc2C(=O)c2cc3ccccc3cc21] |
| A-8 | 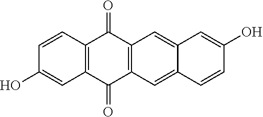 [SMILES: O=C1c2cc(O)ccc2C(=O)c2cc3cc(O)ccc3cc21] |
| A-9 | 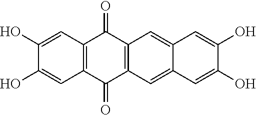 [SMILES: O=C1c2cc(O)c(O)cc2C(=O)c2cc3cc(O)c(O)cc3cc21] |
| A-10 | 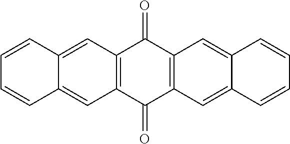 [SMILES: O=C1c2cc3ccccc3cc2C(=O)c2cc3ccccc3cc21] |
| A-11 | 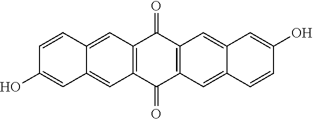 [SMILES: O=C1c2cc3ccc(O)cc3cc2C(=O)c2cc3ccc(O)cc3cc21] |
| A-12 | 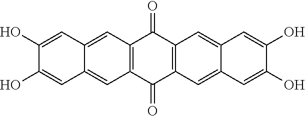 [SMILES: O=C1c2cc3cc(O)c(O)cc3cc2C(=O)c2cc3cc(O)c(O)cc3cc21] |

TABLE 2

| Name | Formula |
|---|---|
| A-13 | 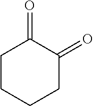 |
| A-14 | 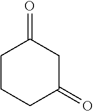 |
| A-15 | 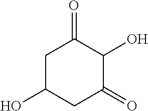 |
| A-16 | 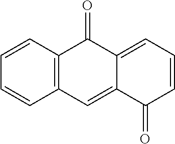 [SMILES: O=C1C=CC=C2C(=O)c3ccccc3C=C12] |
| A-17 | 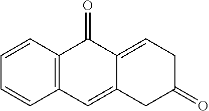 [SMILES: O=C1CC=C2C(=O)c3ccccc3C=C2C1] |
| A-18 | 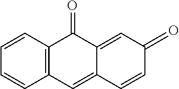 [SMILES: O=C1C=CC2=Cc3ccccc3C(=O)C2=C1] |
| A-19 | 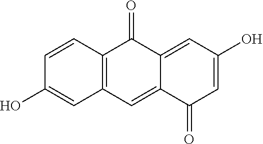 [SMILES: O=C1C=C(O)C=C2C(=O)c3ccc(O)cc3C=C12] |
| A-20 | 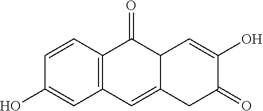 [SMILES: O=C1CC2=Cc3cc(O)ccc3C(=O)C2C=C1O] |
| A-21 | 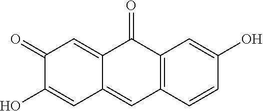 [SMILES: O=C1C=C2C(=O)c3cc(O)ccc3C=C2C=C1O] |
| A-22 | 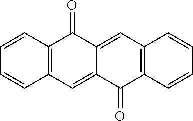 [SMILES: O=C1C2=Cc3ccccc3C(=O)C2=Cc2ccccc21] |
| A-23 | 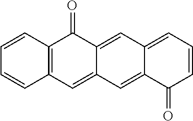 [SMILES: O=C1C=CC=c2cc3c(cc21)=Cc1ccccc1C3=O] |
| A-24 | 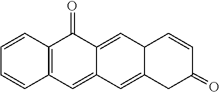 [SMILES: O=C1C=CC2C=C3C(=O)c4ccccc4C=C3C=C2C1] |

TABLE 3

| Name | Formula |
|---|---|
| A-25 | 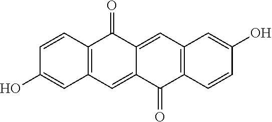 [SMILES: O=C1C2=Cc3cc(O)ccc3C(=O)C2=Cc2cc(O)ccc21] |
| A-26 | 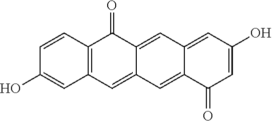 [SMILES: O=C1C=C(O)C=c2cc3c(cc21)=Cc1cc(O)ccc1C3=O] |
| A-27 | 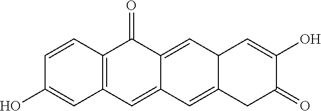 [SMILES: O=C1CC2=CC3=Cc4cc(O)ccc4C(=O)C3=CC2C=C1O] |
| A-28 | 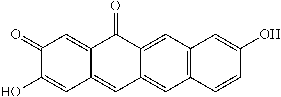 [SMILES: O=C1C=C2C(=O)c3cc4cc(O)ccc4cc3C=C2C=C1O] |
| A-29 | 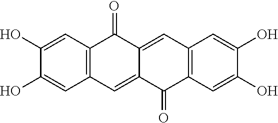 [SMILES: O=C1C2=Cc3cc(O)c(O)cc3C(=O)C2=Cc2cc(O)c(O)cc21] |
| A-30 | 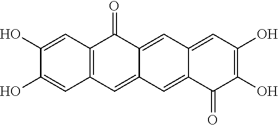 [SMILES: O=C1C(O)=C(O)C=c2cc3c(cc21)=Cc1cc(O)c(O)cc1C3=O] |
| A-31 | 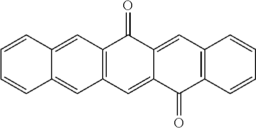 [SMILES: O=C1C2=Cc3cc4ccccc4cc3C(=O)C2=Cc2ccccc21] |
| A-32 | 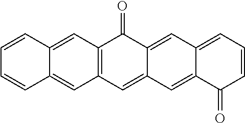 [SMILES: O=C1C=CC=c2cc3c(cc21)=Cc1cc2ccccc2cc1C3=O] |
| A-33 | 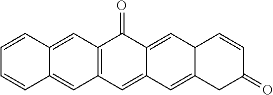 [SMILES: O=C1C=CC2C=C3C(=O)c4cc5ccccc5cc4C=C3C=C2C1] |
| A-34 | 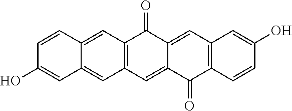 [SMILES: O=C1C2=Cc3cc4cc(O)ccc4cc3C(=O)C2=Cc2cc(O)ccc21] |
| A-35 | 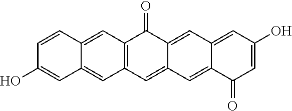 [SMILES: O=C1C=C(O)C=c2cc3c(cc21)=Cc1cc2cc(O)ccc2cc1C3=O] |
| A-36 | 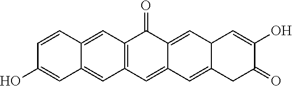 [SMILES: O=C1CC2=CC3=Cc4cc5cc(O)ccc5cc4C(=O)C3=CC2C=C1O] |
| A-37 | 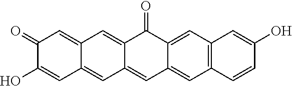 [SMILES: O=C1C=c2cc3c(cc2C=C1O)=Cc1cc2ccc(O)cc2cc1C3=O] |
| A-38 | 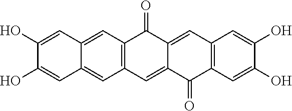 [SMILES: O=C1C2=Cc3cc4cc(O)c(O)cc4cc3C(=O)C2=Cc2cc(O)c(O)cc21] |
| A-39 | 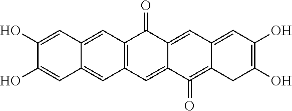 |

TABLE 4

| Name | Formula |
|---|---|
| A-40 | 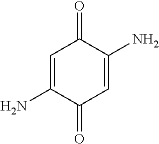 |
| A-41 | 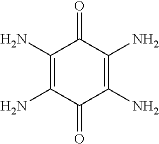 [SMILES: NC1=C(N)C(=O)C(N)=C(N)C1=O] |
| A-42 | 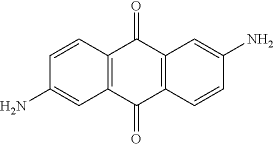 [SMILES: Nc1ccc2c(c1)C(=O)c1ccc(N)cc1C2=O] |
| A-43 | 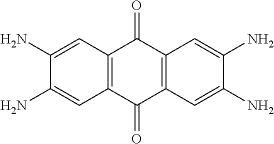 [SMILES: Nc1cc2c(cc1N)C(=O)c1cc(N)c(N)cc1C2=O] |
| A-44 | 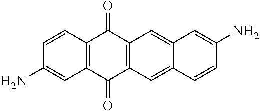 [SMILES: Nc1ccc2c(c1)C(=O)c1cc3ccc(N)cc3cc1C2=O] |
| A-45 | 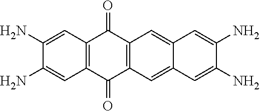 [SMILES: Nc1cc2c(cc1N)C(=O)c1cc3cc(N)c(N)cc3cc1C2=O] |
| A-46 | 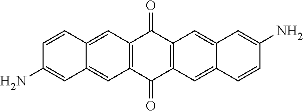 [SMILES: Nc1ccc2cc3c(cc2c1)C(=O)c1cc2ccc(N)cc2cc1C3=O] |
| A-47 | 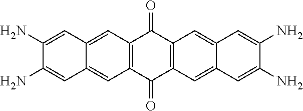 [SMILES: Nc1cc2cc3c(cc2cc1N)C(=O)c1cc2cc(N)c(N)cc2cc1C3=O] |
| A-48 | 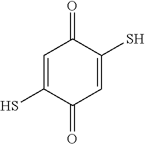 |
| A-49 | 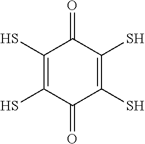 [SMILES: O=C1C(S)=C(S)C(=O)C(S)=C1S] |
| A-50 | 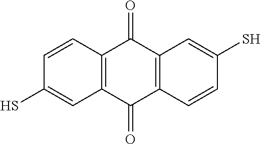 [SMILES: O=C1c2ccc(S)cc2C(=O)c2ccc(S)cc21] |
| A-51 | 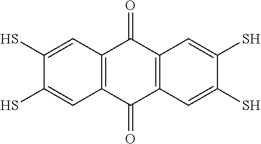 [SMILES: O=C1c2cc(S)c(S)cc2C(=O)c2cc(S)c(S)cc21] |
| A-52 | 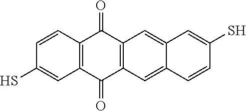 [SMILES: O=C1c2cc(S)ccc2C(=O)c2cc3cc(S)ccc3cc21] |
| A-53 | 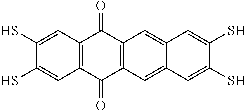 [SMILES: O=C1c2cc(S)c(S)cc2C(=O)c2cc3cc(S)c(S)cc3cc21] |
| A-54 | 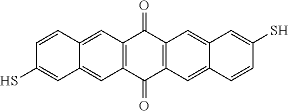 [SMILES: O=C1c2cc3ccc(S)cc3cc2C(=O)c2cc3ccc(S)cc3cc21] |
| A-55 | 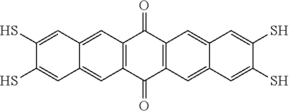 [SMILES: O=C1c2cc3cc(S)c(S)cc3cc2C(=O)c2cc3cc(S)c(S)cc3cc21] |

TABLE 5

| Name | Formula |
|---|---|
| A-56 | 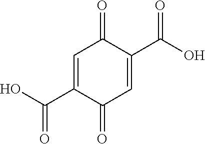 [SMILES: O=C(O)C1=CC(=O)C(C(=O)O)=CC1=O] |
| A-57 | 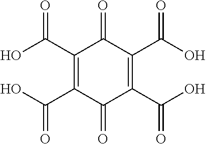 [SMILES: O=C(O)C1=C(C(=O)O)C(=O)C(C(=O)O)=C(C(=O)O)C1=O] |
| A-58 | 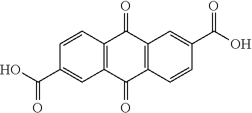 [SMILES: O=C(O)c1ccc2c(c1)C(=O)c1ccc(C(=O)O)cc1C2=O] |
| A-59 | 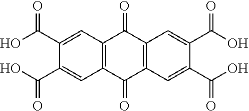 [SMILES: O=C(O)c1cc2c(cc1C(=O)O)C(=O)c1cc(C(=O)O)c(C(=O)O)cc1C2=O] |
| A-60 | 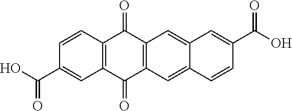 [SMILES: O=C(O)c1ccc2c(c1)C(=O)c1cc3ccc(C(=O)O)cc3cc1C2=O] |
| A-61 | 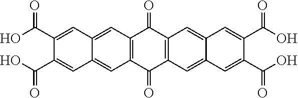 [SMILES: O=C(O)c1cc2cc3c(cc2cc1C(=O)O)C(=O)c1cc2cc(C(=O)O)c(C(=O)O)cc2cc1C3=O] |
| A-62 | 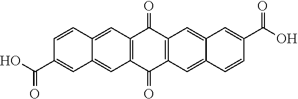 [SMILES: O=C(O)c1ccc2cc3c(cc2c1)C(=O)c1cc2ccc(C(=O)O)cc2cc1C3=O] |
| A-63 | 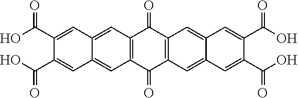 [SMILES: O=C(O)c1cc2cc3c(cc2cc1C(=O)O)C(=O)c1cc2cc(C(=O)O)c(C(=O)O)cc2cc1C3=O] |

### Synthetic Preparation Example 2: Preparation of Monomers B-1 to B-25

TABLE 6

| Name | Formula |
|---|---|
| B-1 | 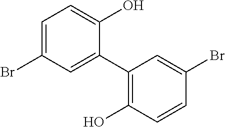 [SMILES: Oc1ccc(Br)cc1-c1cc(Br)ccc1O] |
| B-2 | 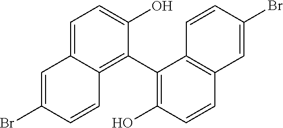 [SMILES: Oc1ccc2cc(Br)ccc2c1-c1c(O)ccc2cc(Br)ccc12] |
| B-3 | 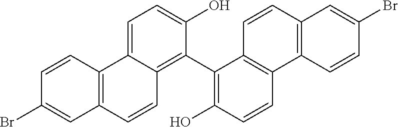 [SMILES: Oc1ccc2c(ccc3cc(Br)ccc32)c1-c1c(O)ccc2c1ccc1cc(Br)ccc12] |
| B-4 | 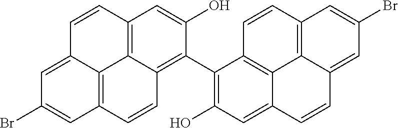 [SMILES: Oc1cc2ccc3cc(Br)cc4ccc(c1-c1c(O)cc5ccc6cc(Br)cc7ccc1c5c67)c2c34] |
| B-5 | 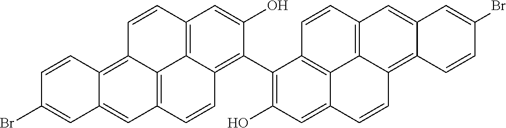 [SMILES: Oc1cc2ccc3c4ccc(Br)cc4cc4ccc(c1-c1c(O)cc5ccc6c7ccc(Br)cc7cc7ccc1c5c76)c2c43] |
| B-6 | 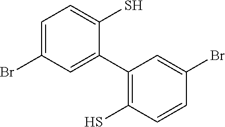 [SMILES: Sc1ccc(Br)cc1-c1cc(Br)ccc1S] |
| B-7 | 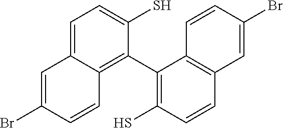 [SMILES: Sc1ccc2cc(Br)ccc2c1-c1c(S)ccc2cc(Br)ccc12] |
| B-8 | 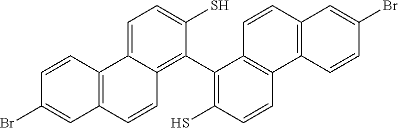 [SMILES: Sc1ccc2c(ccc3cc(Br)ccc32)c1-c1c(S)ccc2c1ccc1cc(Br)ccc12] |
| B-9 | 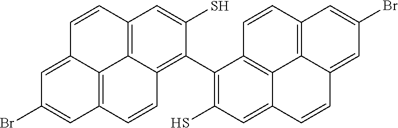 [SMILES: Sc1cc2ccc3cc(Br)cc4ccc(c1-c1c(S)cc5ccc6cc(Br)cc7ccc1c5c67)c2c34] |
| B-10 | 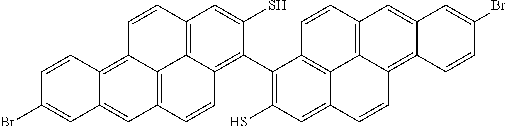 [SMILES: Sc1cc2ccc3c4ccc(Br)cc4cc4ccc(c1-c1c(S)cc5ccc6c7ccc(Br)cc7cc7ccc1c5c76)c2c43] |
| B-11 | 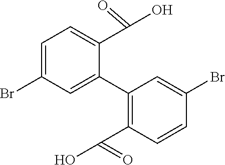 [SMILES: O=C(O)c1ccc(Br)cc1-c1cc(Br)ccc1C(=O)O] |
| B-12 | 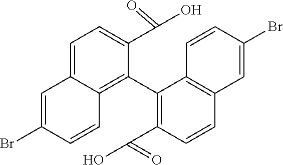 [SMILES: O=C(O)c1ccc2cc(Br)ccc2c1-c1c(C(=O)O)ccc2cc(Br)ccc12] |
| B-13 | 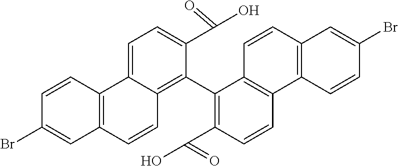 [SMILES: O=C(O)c1ccc2c(ccc3cc(Br)ccc32)c1-c1c(C(=O)O)ccc2c1ccc1cc(Br)ccc12] |
| B-14 | 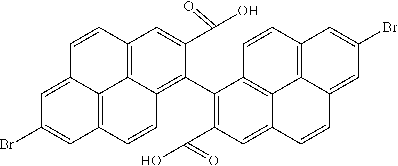 [SMILES: O=C(O)c1cc2ccc3cc(Br)cc4ccc(c1-c1c(C(=O)O)cc5ccc6cc(Br)cc7ccc1c5c67)c2c34] |
| B-15 | 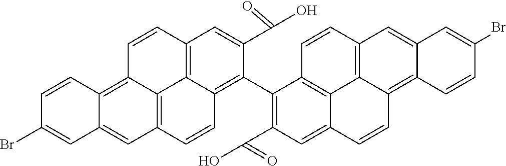 [SMILES: O=C(O)c1cc2ccc3c4ccc(Br)cc4cc4ccc(c1-c1c(C(=O)O)cc5ccc6c7ccc(Br)cc7cc7ccc1c5c76)c2c43] |
| B-16 | 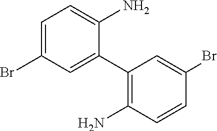 [SMILES: Nc1ccc(Br)cc1-c1cc(Br)ccc1N] |
| B-17 | 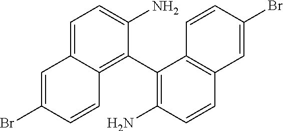 [SMILES: Nc1ccc2cc(Br)ccc2c1-c1c(N)ccc2cc(Br)ccc12] |
| B-18 | 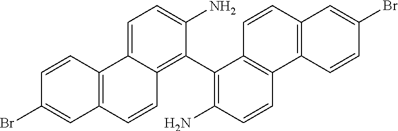 [SMILES: Nc1ccc2c(ccc3cc(Br)ccc32)c1-c1c(N)ccc2c1ccc1cc(Br)ccc12] |
| B-19 | 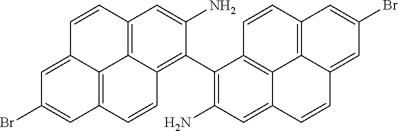 [SMILES: Nc1cc2ccc3cc(Br)cc4ccc(c1-c1c(N)cc5ccc6cc(Br)cc7ccc1c5c67)c2c34] |
| B-20 | 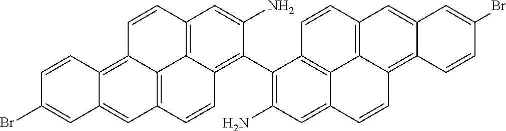 [SMILES: Nc1cc2ccc3c4ccc(Br)cc4cc4ccc(c1-c1c(N)cc5ccc6c7ccc(Br)cc7cc7ccc1c5c76)c2c43] |
| B-21 | 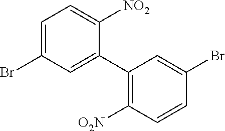 [SMILES: O=[N+]([O-])c1ccc(Br)cc1-c1cc(Br)ccc1[N+](=O)[O-]] |
| B-22 | 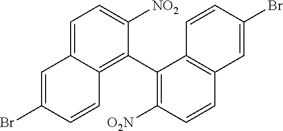 [SMILES: O=[N+]([O-])c1ccc2cc(Br)ccc2c1-c1c([N+](=O)[O-])ccc2cc(Br)ccc12] |
| B-23 | 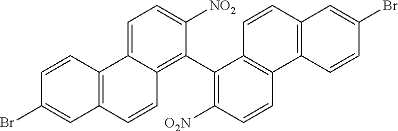 [SMILES: O=[N+]([O-])c1ccc2c(ccc3cc(Br)ccc32)c1-c1c([N+](=O)[O-])ccc2c1ccc1cc(Br)ccc12] |
| B-24 | 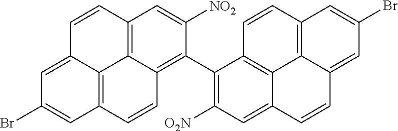 [SMILES: O=[N+]([O-])c1cc2ccc3cc(Br)cc4ccc(c1-c1c([N+](=O)[O-])cc5ccc6cc(Br)cc7ccc1c5c67)c2c34] |
| B-25 | 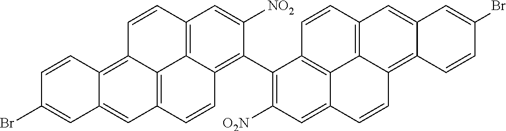 [SMILES: O=[N+]([O-])c1cc2ccc3c4ccc(Br)cc4cc4ccc(c1-c1c([N+](=O)[O-])cc5ccc6c7ccc(Br)cc7cc7ccc1c5c76)c2c43] |

### Synthetic Preparation Example 3: Preparation of Monomers C-1 to C-20

TABLE 7

| Name | Formula |
|---|---|
| C-1 | 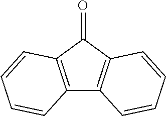 [SMILES: O=C1c2ccccc2-c2ccccc21] |
| C-2 | 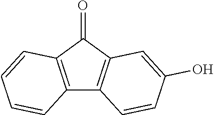 [SMILES: O=C1c2ccccc2-c2ccc(O)cc21] |
| C-3 | 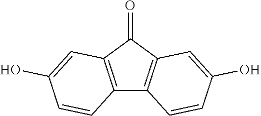 [SMILES: O=C1c2cc(O)ccc2-c2ccc(O)cc21] |
| C-4 | 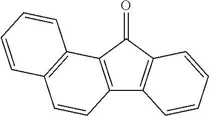 [SMILES: O=C1c2ccccc2-c2ccc3ccccc3c21] |
| C-5 | 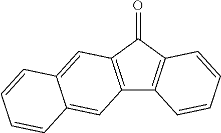 [SMILES: O=C1c2ccccc2-c2cc3ccccc3cc21] |
| C-6 | 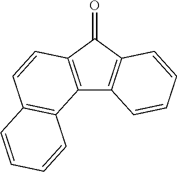 [SMILES: O=C1c2ccccc2-c2c1ccc1ccccc21] |
| C-7 | 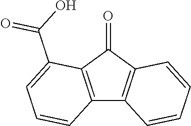 [SMILES: O=C(O)c1cccc2c1C(=O)c1ccccc1-2] |
| C-8 | 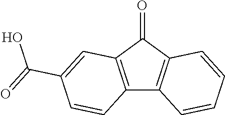 [SMILES: O=C(O)c1ccc2c(c1)C(=O)c1ccccc1-2] |
| C-9 | 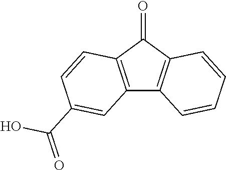 [SMILES: O=C(O)c1ccc2c(c1)-c1ccccc1C2=O] |
| C-10 | 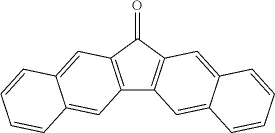 [SMILES: O=C1c2cc3ccccc3cc2-c2cc3ccccc3cc21] |
| C-11 | 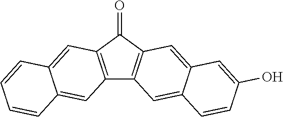 [SMILES: O=C1c2cc3ccccc3cc2-c2cc3ccc(O)cc3cc21] |
| C-12 | 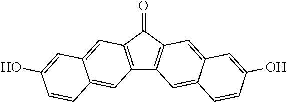 [SMILES: O=C1c2cc3cc(O)ccc3cc2-c2cc3ccc(O)cc3cc21] |
| C-13 | 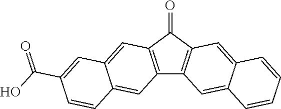 [SMILES: O=C(O)c1ccc2cc3c(cc2c1)C(=O)c1cc2ccccc2cc1-3] |

TABLE 8

| Name | Formula |
|---|---|
| C-14 | 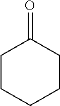 |
| C-15 | 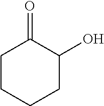 |
| C-16 | 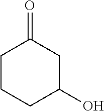 |
| C-17 | 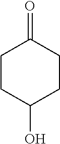 |
| C-18 | 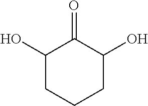 |
| C-19 | 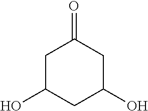 |
| C-20 | 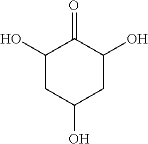 |

### Synthesis Example: Synthesis of Compound

### Example 1

Under a nitrogen atmosphere, a compound (20 g) represented by B-3 and tetrahydrofuran (100 ml) were added into a three-necked flask (500 ml) equipped with a thermometer and a reflux tube and then stirred to prepare a first uniform mixture solution. The first mixture solution was cooled to a temperature of −20° C. and an n-butyllithium-hexane solution (30 ml at a molar concentration of 2.5 mol/L) was slowly added dropwise to the first mixture solution for 30 minutes.

A solution of a compound (18 g) represented by Formula A-4 dissolved in tetrahydrofuran (100 ml) was slowly added dropwise to the first mixture solution, to which the n-butyllithium-hexane solution was added dropwise, for an hour to prepare a mixture. The mixture was heated to room temperature and then stirred at room temperature for 24 hours. The stirred mixture was aged for an hour and then, the aged mixture was re-cooled to −20° C. The n-butyllithium-hexane solution (10 ml at a molar concentration of 2.5 mol/L) was slowly added dropwise to the re-cooled mixture for 10 minutes.

The above mixture, to which the n-butyllithium-hexane solution was added dropwise, was rapidly heated to room temperature and then, a solution of a compound (3 g) represented by Formula C-1 dissolved in tetrahydrofuran (100 ml) was swiftly added dropwise to the mixture. The mixture, to which the compound (3 g) represented by Formula C-1 was added, was aged at room temperature for an hour and then, the aged mixture was diluted with ethyl acetate (400 ml). The diluted mixture was transferred to a separatory funnel, and liquid separation and water-washing were performed until an aqueous layer became neutral. After recovering an organic layer, the solvent was removed by a distillation method to obtain a suspension. The obtained suspension was added dropwise to methanol (300 g) and then stirred to precipitate crystals. The crystals were dried in vacuo at a temperature of 60° C. to finally synthesize a compound of Example 1, the compound represented by Formula 1-1 having a weight average molecular weight of 4,200 g/mol (the number of repeating units: n=7).

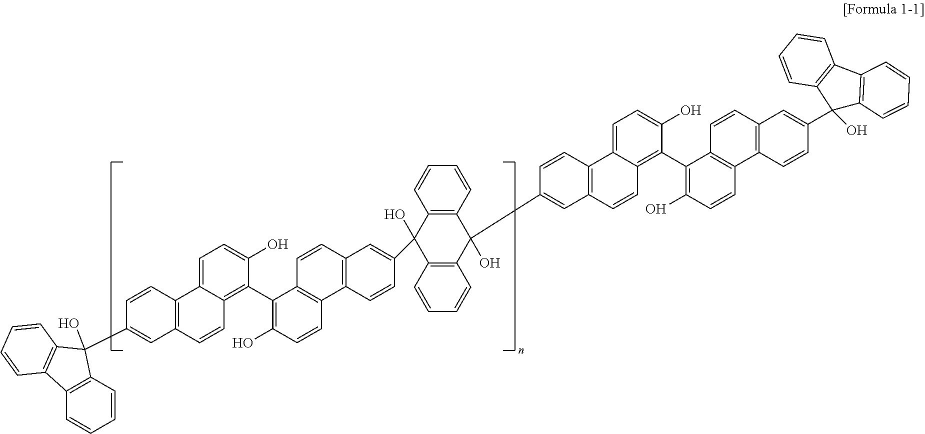

### Example 2

A compound was synthesized in the same manner as in Example 1, but a compound (15 g) represented by Formula B-4 was used instead of the compound (20 g) represented by Formula B-3. In addition, a mixture was heated to room temperature and then stirred at room temperature for 20 hours instead 24 hours to finally synthesize the compound of Example 2, the compound represented by Formula 1-2 having a weight average molecular weight of 3,500 g/mol (the number of repeating units: n=5).

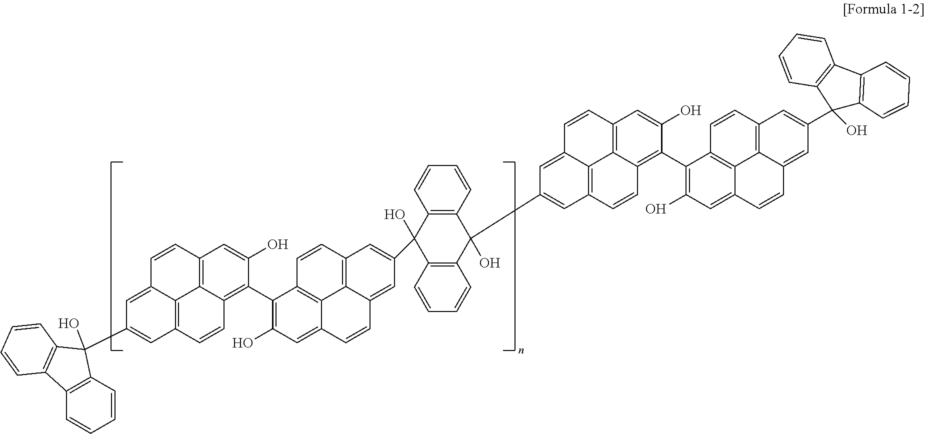

### Example 3

A compound was synthesized in the same manner as in Example 1, but a compound (18 g) represented by Formula B-18 was used instead of the compound (20 g) represented by Formula B-3. In addition, a mixture was heated to room temperature and then stirred at room temperature for 13 hours instead of 24 hours to finally synthesize the compound of Example 3, the compound represented by Formula 1-3 having a weight average molecular weight of 2,200 g/mol (the number of repeating units: n=3).

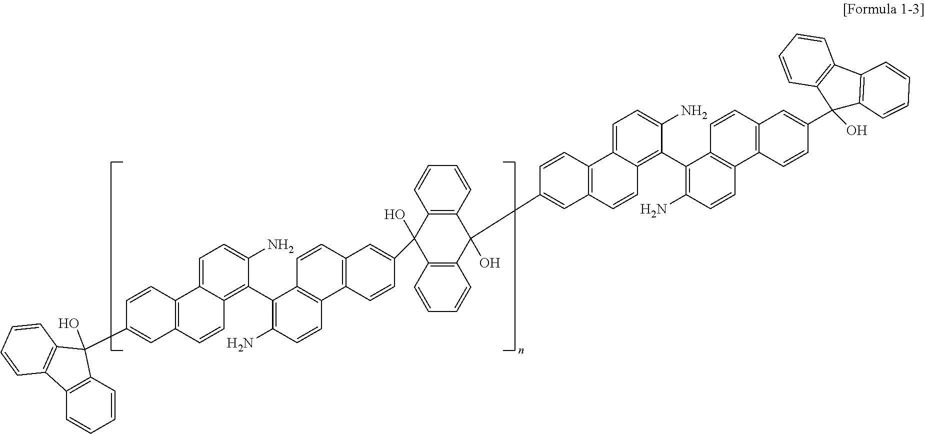

### Example 4

A compound was synthesized in the same manner as in Example 1, but a compound (13 g) represented by Formula B-19 was used instead of the compound (20 g) represented by Formula B-3. In addition, a mixture was heated to room temperature and then stirred at room temperature for 12 hours instead of 24 hours to finally synthesize the compound of Example 4, the compound represented by Formula 1-4 having a weight average molecular weight of 1,600 g/mol (the number of repeating units: n=2).

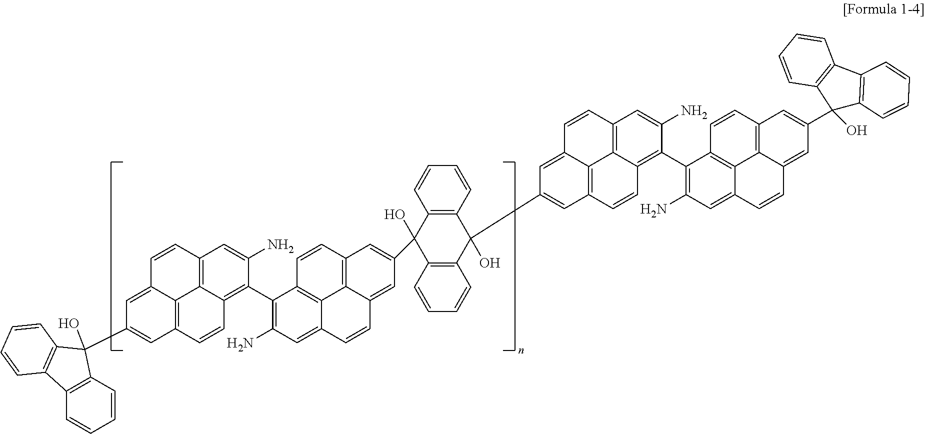

### Example 5

A compound was synthesized in the same manner as in Example 1, but a compound (13 g) represented by Formula B-1, a compound (15 g) represented by Formula A-3, and a compound (4 g) represented by Formula C-3 were used instead of the compound (20 g) represented by Formula B-3, the compound (18 g) represented by Formula A-4, and the compound (3 g) represented by Formula C-1, respectively. In addition, a mixture was heated to room temperature and then stirred at room temperature for 34 hours instead of 24 hours to finally synthesize the compound of Example 5, the compound represented by Formula 1-5 and having a weight average molecular weight of 6,000 g/mol (the number of repeating units: n=15).

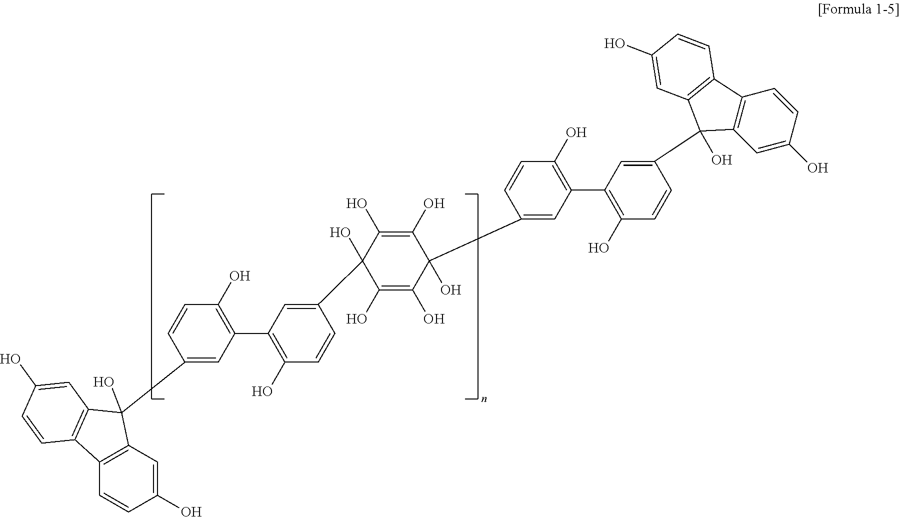

### Example 6

A compound was synthesized in the same manner as in Example 1, but a compound (26 g) represented by Formula B-5, a compound (27 g) represented by Formula A-10, and a compound (5 g) represented by Formula C-10 were used instead of the compound (20 g) represented by Formula B-3, the compound (18 g) represented by Formula A-4, and the compound (3 g) represented by Formula C-1, respectively. In addition, a mixture was heated to room temperature and then stirred at room temperature for 14 hours instead of 24 hours at room temperature to finally synthesize the compound of Example 6, the compound represented by Formula 1-6 having a weight average molecular weight of 2,500 g/mol (the number of repeating units: n=2).

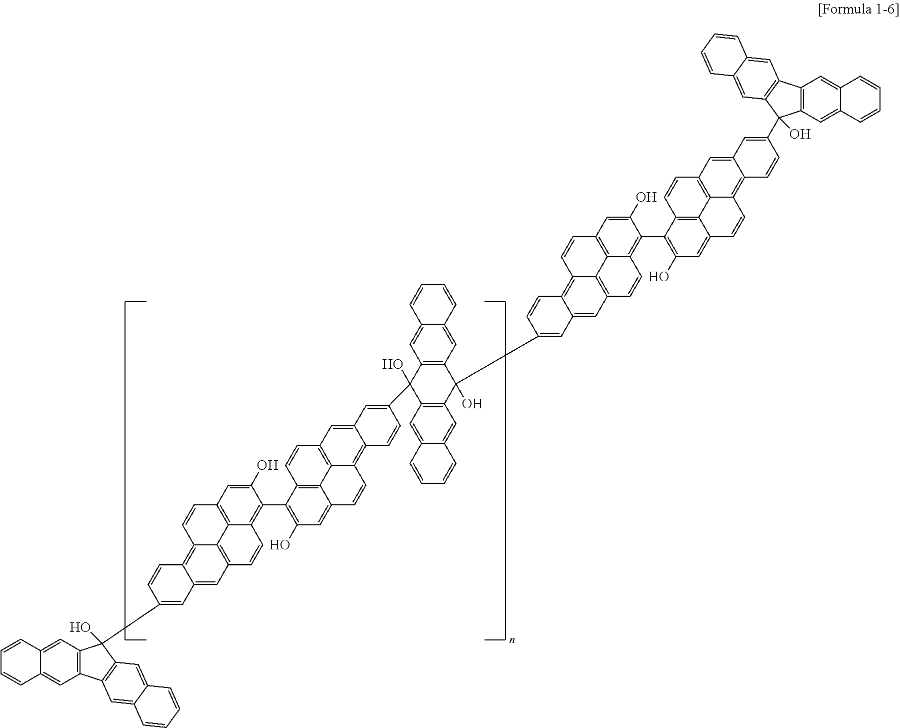

### Example 7

A compound was synthesized in the same manner as in Example 1, but a compound (24 g) represented by Formula A-6 was used instead of the compound (18 g) represented by Formula A-4. In addition, a mixture was heated to room temperature and then stirred at room temperature for 19 hours instead of 24 hours to finally synthesize the compound of Example 7, the compound represented by Formula 1-7 having a weight average molecular weight of 3,400 g/mol (the number of repeating units: n=4).

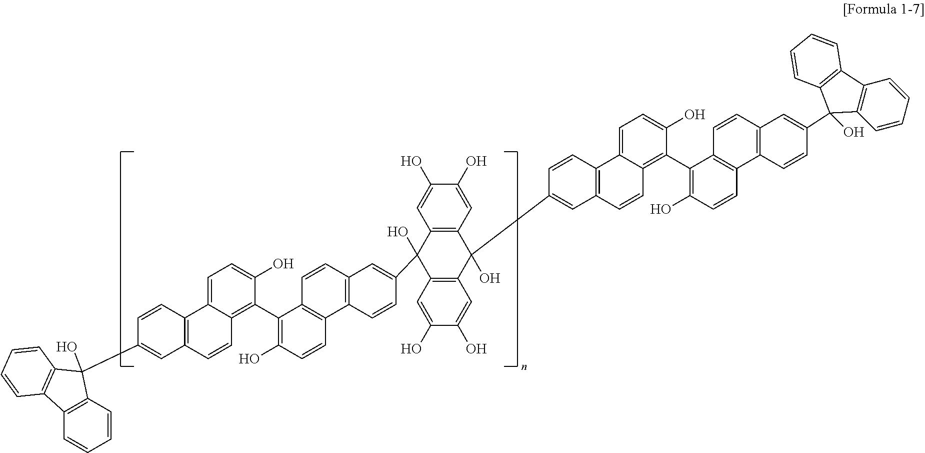

### Example 8

A compound was synthesized in the same manner as in Example 1, but a compound (24 g) represented by Formula A-6 and a compound (4 g) represented by Formula C-3 were used instead of the compound (18 g) represented by Formula A-4 and the compound (3 g) represented by Formula C-1, respectively. In addition, a mixture was heated to room temperature and then stirred at room temperature for 19 hours instead of 24 hours to finally synthesize the compound of Example 8, the compound represented by Formula 1-8 having a weight average molecular weight of 3,400 g/mol (the number of repeating units: n=4).

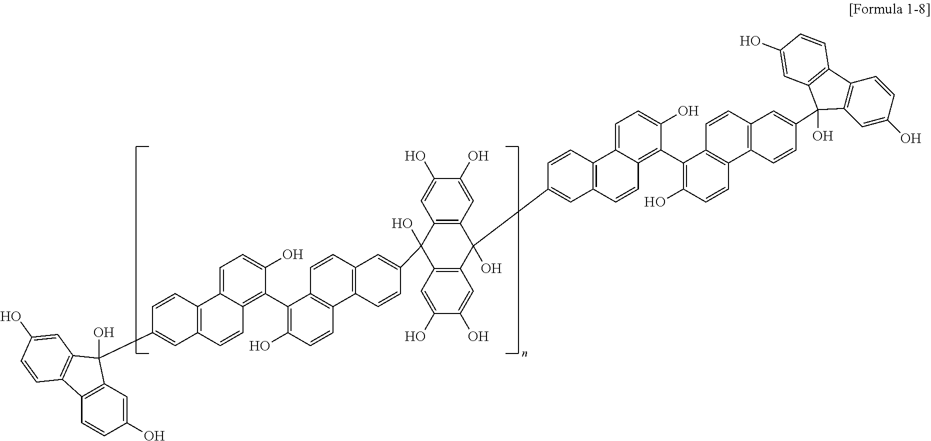

### Example 9

A compound was synthesized in the same manner as in Example 1, but cyclohexanone (3 g) was used instead of the compound (3 g) represented by Formula C-1 to finally synthesize the compound of Example 9, the compound represented by Formula 1-9 having a weight average molecular weight of 4,000 g/mol (the number of repeating units: n=6).

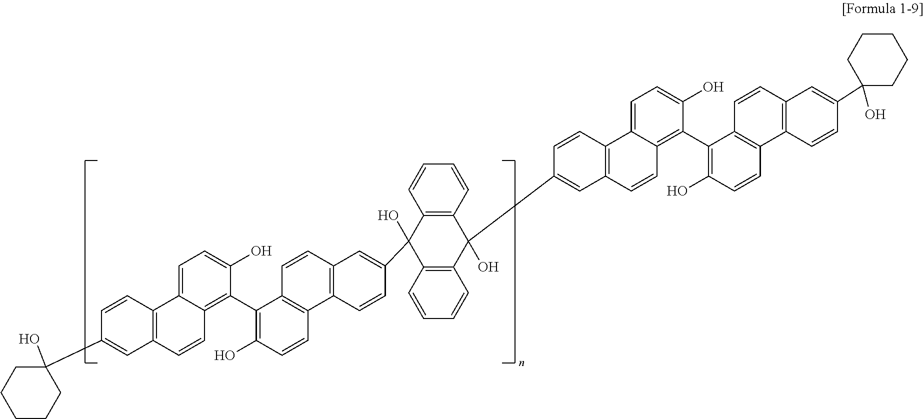

### Example 10

A compound was synthesized in the same manner as in Example 1, but a compound (23 g) represented by Formula A-7 was used instead of the compound (18 g) represented by Formula A-4. In addition, a mixture was heated to room temperature and then stirred at room temperature for 17 hours instead of 24 hours to finally synthesize the compound of Example 10, the compound represented by Formula 1-10 having a weight average molecular weight of 3,000 g/mol (the number of repeating units: n=4).

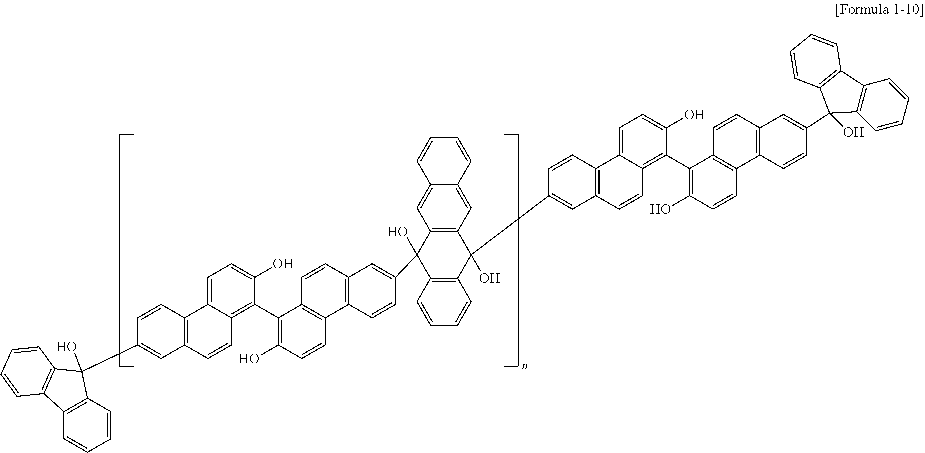

### Example 11

A compound was synthesized in the same manner as in Example 1, but a compound (22 g) represented by Formula B-4 and a compound (23 g) represented by Formula A-7 were used instead of the compound (20 g) represented by Formula B-3 and the compound (18 g) represented by Formula A-4, respectively. In addition, a mixture was heated to room temperature and then stirred at room temperature for 15 hours instead of 24 hours to finally synthesize the compound of Example 11, the compound represented by Formula 1-11 having a weight average molecular weight of 2,500 g/mol (the number of repeating units: n=3).

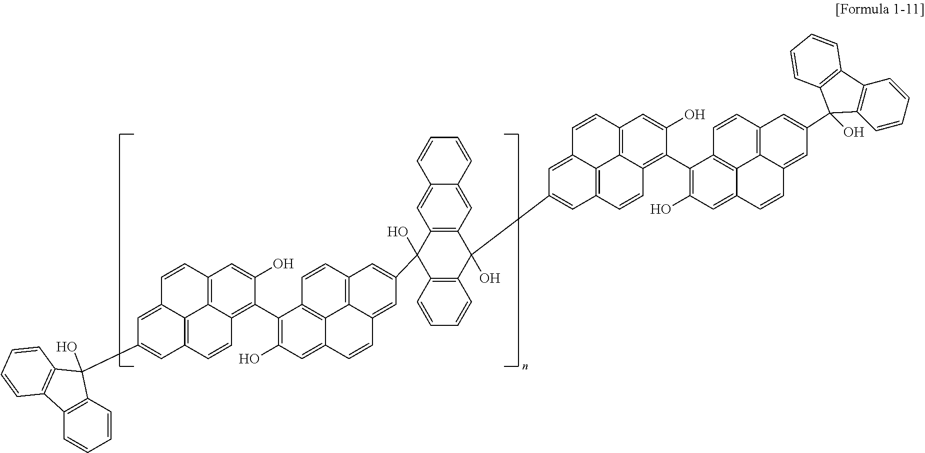

### Example 12

A compound was synthesized in the same manner as in Example 1, but a compound (21 g) represented by Formula A-5 was used instead of the compound (18 g) represented by Formula A-4. In addition, a mixture was heated to room temperature and then stirred at room temperature for 23 hours instead of 24 hours to finally synthesize the compound of Example 12, the compound represented by Formula 1-12 having a weight average molecular weight of 4,000 g/mol (the number of repeating units: n=6).

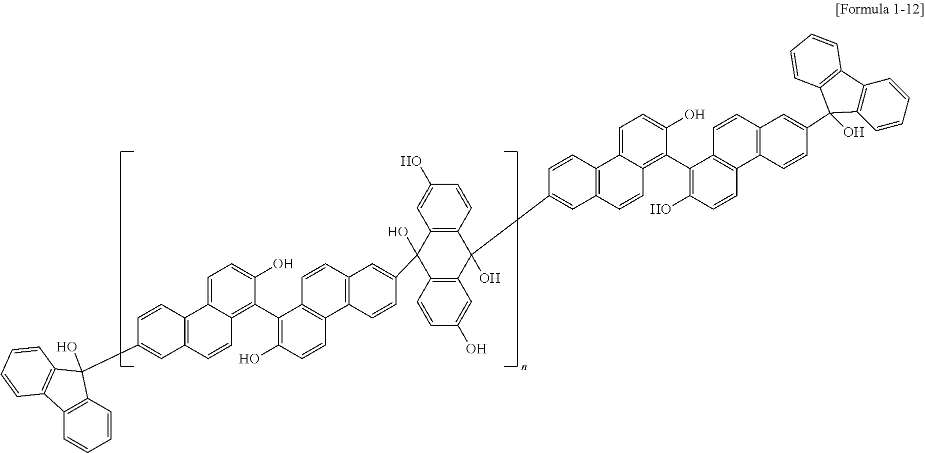

### Example 13

A compound was synthesized in the same manner as in Example 1, but a compound (22 g) represented by Formula B-4 and a compound (21 g) represented by Formula A-5 were used instead of the compound (20 g) represented by Formula B-3 and the compound (18 g) represented by Formula A-4, respectively. In addition, a mixture was heated to room temperature and then stirred at room temperature for 19 hours instead of 24 hours to finally synthesize the compound of Example 13, the compound represented by Formula 1-13 having a weight average molecular weight of 3,300 g/mol (the number of repeating units: n=4).

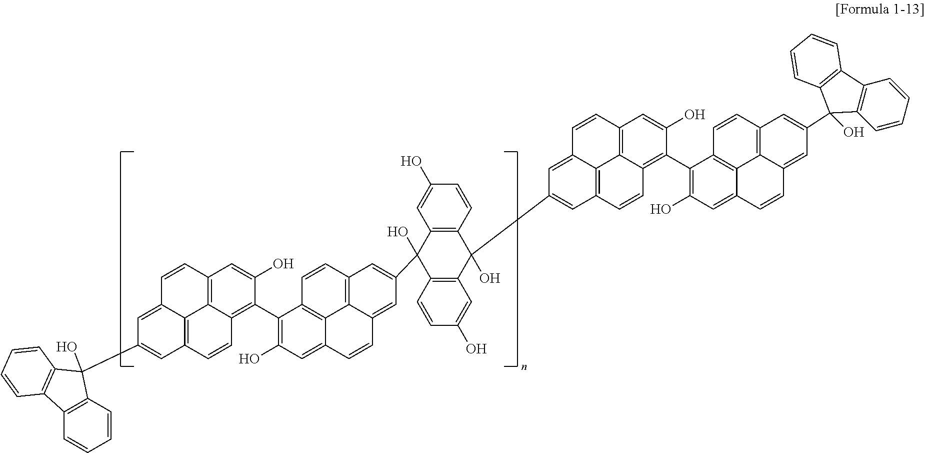

### Example 14

Under a nitrogen atmosphere, a compound (15 g) represented by B-3 and tetrahydrofuran (75 ml) were added into a three-necked flask (500 ml) equipped with a thermometer and a reflux tube and then stirred to prepare a first uniform mixture solution. The first mixture solution was cooled to a temperature of −20° C. and an n-butyllithium-hexane solution (30 ml at a molar concentration of 2.5 mol/L) was slowly added dropwise to the first mixture solution for 30 minutes.

A solution of a compound (18 g) represented by Formula A-4 dissolved in tetrahydrofuran (100 ml) was slowly added dropwise to the first mixture solution, to which the n-butyllithium-hexane solution was added dropwise, for an hour to prepare a mixture. The mixture was heated to room temperature and then stirred at room temperature for 22 hours. The stirred mixture was aged for an hour to prepare the aged mixture. A mixture solution of a compound (5 g) represented by Formula B-4 and tetrahydrofuran (25 ml) was swiftly added dropwise to the aged mixture, and then additionally stirred for an hour. The mixture which was additionally stirred for an hour was re-cooled to a temperature of −20° C. and then the n-butyllithium-hexane solution (10 ml at a molar concentration of 2.5 mol/L) was added dropwise to the re-cooled mixture for 10 minutes.

The above mixture, to which the n-butyllithium-hexane solution was added dropwise, was rapidly heated to room temperature and then, a solution of a compound (3 g) represented by Formula C-1 dissolved in tetrahydrofuran (10 ml) was swiftly added dropwise to the mixture. The mixture, to which the compound (3 g) represented by Formula C-1 was added, was aged at room temperature for an hour and then, the aged mixture was diluted with ethyl acetate (400 ml). The diluted mixture was transferred to a separatory funnel, and then, liquid separation and water-washing were performed until an aqueous layer became neutral. After recovering an organic layer, the solvent was removed by a distillation method to obtain a suspension. The obtained suspension was added dropwise to methanol (300 g) and then stirred to precipitate crystals. The crystals were dried in vacuo at a temperature of 60° C. to finally synthesize a compound of Example 14, the compound represented by Formula 1-14 having a weight average molecular weight of 3,800 g/mol (the number of repeating units: n=6).

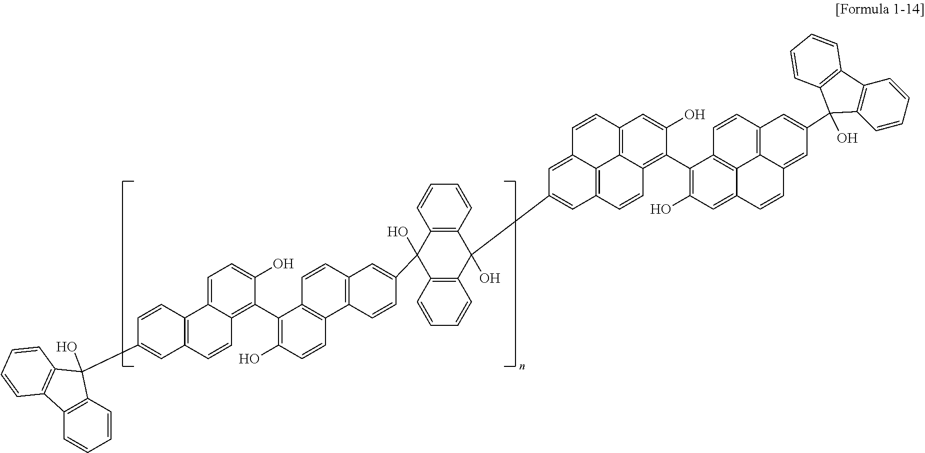

### Example 15

A compound was synthesized in the same manner as in Example 14, but cyclohexanone (3 g) was used instead of the compound (3 g) represented by Formula C-1 to finally synthesize the compound of Example 15, the compound represented by Formula 1-15 having a weight average molecular weight of 3,700 g/mol (the number of repeating units: n=6).

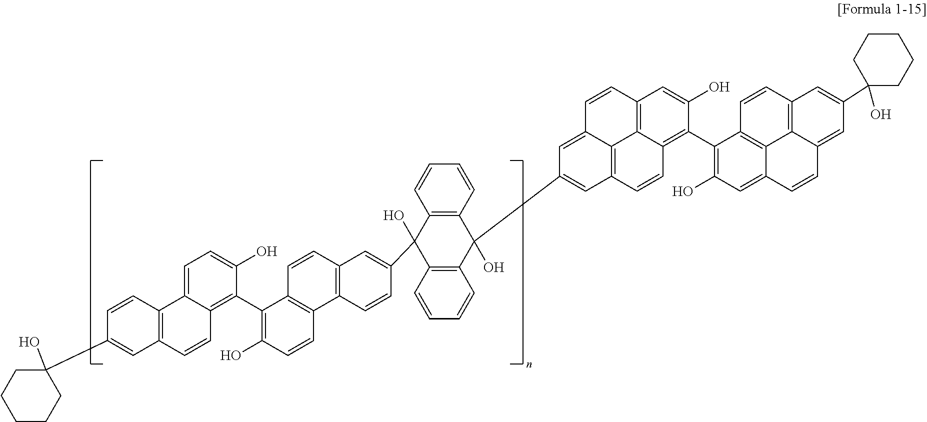

### Example 16

A compound was synthesized in the same manner as in Example 1, but a compound (18 g) represented by Formula B-1 was used instead of the compound (20 g) represented by Formula B-3. In addition, a mixture was heated to room temperature and then stirred at room temperature for 27 hours instead of 24 hours to finally synthesize the compound of Example 16, the compound represented by Formula 1-16 and having a weight average molecular weight of 4,650 g/mol (the number of repeating units: n=11).

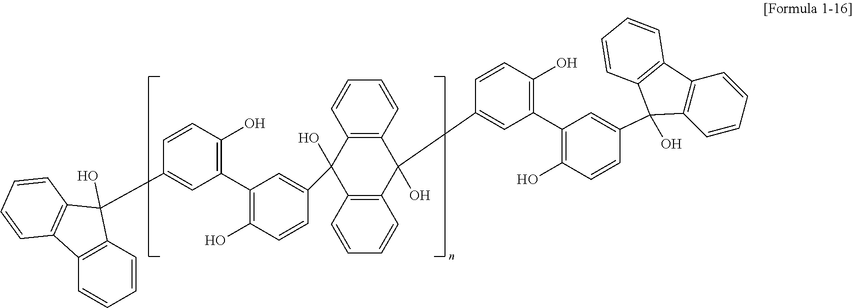

### Reference Example 1: Compound Having Weight Average Molecular Weight of 970 g/Mol Unlike Example 5

A compound was synthesized in the same manner as in Example 5, but a mixture was heated to room temperature and then stirred at room temperature for 5 hours instead of 34 hours to finally synthesize the compound of Reference Example 1 having a weight average molecular weight of 970 g/mol (the number of repeating units: n=1).

### Reference Example 2: Compound Having Weight Average Molecular Weight of 15,000 g/Mol Unlike Example 5

A compound was synthesized in the same manner as in Example 5, but a mixture was heated to room temperature and then stirred at room temperature for 78 hours instead of 34 hours to finally synthesize the compound of Reference Example 2 having a weight average molecular weight of 15,000 g/mol (the number of repeating units: n=40).

### Comparative Example 1

1-Hydroxypyrene (21.83 g), 4,4′-oxybis((methoxymethyl)benzene) (25.81 g), diethyl sulfate (1.23 g), and propylene glycol monomethyl ether acetate (PGMEA, 32.58 g) were put into a flask and then stirred at a temperature of 100° C. for 12 hours to perform a polymerization reaction. The reaction was terminated when a reaction product had a weight average molecular weight of 3,500 g/mol. When the polymerization reaction was terminated, the reaction product was gradually cooled to room temperature and then, the cooled product was added to a mixture of 40 g of distilled water and 400 g of methanol, stirred strongly, and allowed to stand still. Next, a first process was performed by removing a supernatant therefrom and by dissolving a precipitate therein in cyclohexanone (80 g), followed by strongly stirring the resulting solution with methanol (320 g). Then, a second process was performed by removing the obtained supernatant therefrom once again and by dissolving a precipitate therein in cyclohexanone (80 g). When the first and second processes are regarded as one purification process, such a purification process was performed three times in total. The purified polymer was dissolved in cyclohexanone (80 g) and then, the methanol and distilled water remaining in the resulting solution were removed under a reduced pressure to synthesize a compound represented by Formula 2-1 (the number of repeating units: n=9).

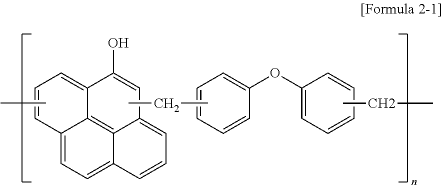

### Comparative Example 2

6,6′-(9H-fluorene-9,9-diyl)dinaphthalen-2-ol (22.53 g), 4,4′-oxybis((methoxymethyl)benzene) (12.91 g), diethyl sulfate (0.77 g), and propylene glycol monomethyl ether acetate (59.07 g) were put into a flask and then stirred at a temperature of 100° C. for 12 hours to perform a polymerization reaction. The reaction was terminated when a reaction product had a weight average molecular weight of 3,500 g/mol. When the polymerization reaction was terminated, the reaction product was gradually cooled to room temperature and then, the cooled product was added to a mixture of 40 g of distilled water and 400 g of methanol, stirred strongly, and allowed to stand still. Next, a first process was performed by removing a supernatant therefrom and by dissolving a precipitate therein in cyclohexanone (80 g), followed by strongly stirring the resulting solution with methanol (320 g). Then, a second process was performed by removing the obtained supernatant therefrom once again and by dissolving a precipitate therein in cyclohexanone (80 g). When the first and second processes are regarded as one purification process, such a purification process was performed three times in total. The purified polymer was dissolved in cyclohexanone (80 g) and then the methanol and distilled water remaining in the resulting solution were removed under a reduced pressure to synthesize a compound represented by Formula 2-2 (the number of repeating units: n=6).

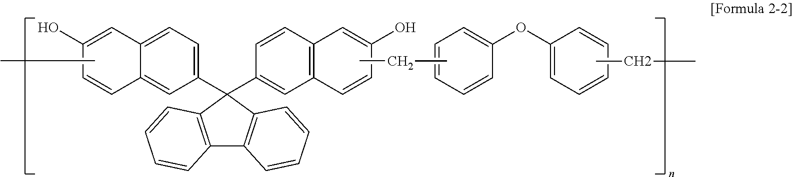

### Comparative Example 3

Under a nitrogen atmosphere, 1-bromopyrene (13 g) and tetrahydrofuran (130 ml) were added to a three-necked flask (500 ml) equipped with a thermometer and a reflux tube and then uniformly stirred. Next, the flask was cooled with dry ice, and an n-butyllithium-hexane solution (20 ml at a molar concentration of 2.5 mol/L) was added dropwise for 20 minutes, followed by aging for 5 hours. When the aging was completed, a compound (33.0 g) of 2,6-dihydroxyanthracene-9,10-dione was dissolved in tetrahydrofuran to prepare a 20% by weight of a reaction solution. Then, a refrigerant was changed to an ice bath at a temperature of −20° C. and the reaction solution was added dropwise to the aged resultant for 20 minutes. The product was aged at room temperature for 5 hours and then the reaction was terminated with a saturated aqueous ammonium chloride solution (70 ml). The resulting product was diluted with ethyl acetate (250 ml) and transferred to a separatory funnel. Then, liquid separation and water-washing were performed until an aqueous layer became neutral. After recovering an organic layer, the solvent was removed by a distillation method to obtain a suspension. Heptane (200 g) was added dropwise thereto and then stirred to precipitate crystals. The crystals were collected by filtration with a funnel, washed three times with a washing solution (200 ml at a volume ratio of ethyl acetate to hexane of 1:2 (v/v)), and dried in vacuo at a temperature of 60° C. to synthesize a compound represented by Formula 2-3.

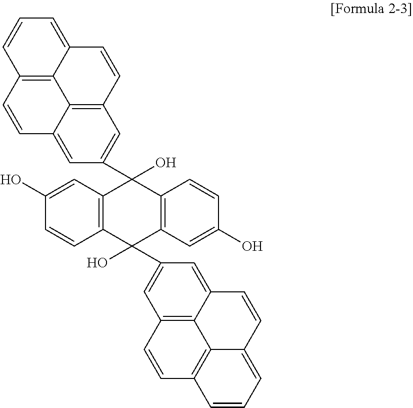 [SMILES: Oc1ccc2c(c1)C(O)(c1cc3ccc4cccc5ccc(c1)c3c45)c1ccc(O)cc1C2(O)c1cc2ccc3cccc4ccc(c1)c2c34]

### Preparation Example 1: Preparation of Resist Underlayer Film Composition

Resist underlayer film compositions were prepared according to the following Examples and Comparative Examples.

### Examples 1, 2, 5, and 7 to 16

Each of compounds prepared according to Examples 1, 2, 5, and 7 to 16 was dissolved in propylene glycol monomethyl ether acetate (PGMEA) and then filtered through a 0.45-μm syringe filter to prepare each of resist underlayer film compositions. The compound contained in the resist underlayer film composition was adjusted to account for 2% to 20% by weight with respect to a total weight of the resist underlayer film composition.

### Examples 3, 4, and 6

Each of compounds prepared according to Examples 3, 4, and 6 was dissolved in a solvent mixture in which propylene glycol monomethyl ether acetate and cyclohexanone were mixed in a volume ratio of 5:5 (v/v), and then filtered through a 0.45-μm syringe filter to prepare each of resist underlayer film compositions. The compound contained in the resist underlayer film composition was adjusted to account for 2% to 20% by weight with respect to a total weight of the resist underlayer film composition.

### Comparative Example 1

A compound prepared according to Comparative Example 1 was dissolved in a solvent mixture in which propylene glycol monomethyl ether acetate and cyclohexanone were mixed in a volume ratio of 7:3 (v/v), and then filtered through a 0.45-μm syringe filter to prepare a resist underlayer film composition. The compound contained in the resist underlayer film composition was adjusted to account for 2% to 15% by weight with respect to a total weight of the resist underlayer film composition.

### Comparative Example 2

A compound prepared according to Comparative Example 2 was dissolved in a solvent mixture in which propylene glycol monomethyl ether acetate and cyclohexanone were mixed in a volume ratio of 7:3 (v/v), and then filtered through a 0.45-μm syringe filter to prepare a resist underlayer film composition. The compound contained in the resist underlayer film composition was adjusted to account for 2% to 20% by weight with respect to a total weight of the resist underlayer film composition.

### Comparative Example 3

A compound prepared according to Comparative Example 3 was dissolved in a solvent mixture in which propylene glycol monomethyl ether acetate and cyclohexanone were mixed in a volume ratio of 5:5 (v/v), and then filtered through a 0.45-μm syringe filter to prepare a resist underlayer film composition. The compound contained in the resist underlayer film composition was adjusted to account for 2% to 30% by weight with respect to a total weight of the resist underlayer film composition.

### Reference Examples 1 and 2

Each of compounds prepared according to Reference Examples 1 and 2 was dissolved in propylene glycol monomethyl ether acetate (PGMEA) and then filtered through a 0.45-μm syringe filter to prepare each of resist underlayer film compositions. The compound contained in the resist underlayer film composition was adjusted to account for 2% to 20% by weight with respect to a total weight of the resist underlayer film composition.

### Experimental Example 1-1: Evaluation of Etching Resistance

The resist underlayer film compositions (4 g) prepared according to Preparation Example 1 were each independently spin-coated on a silicon wafer and then baked in a high-temperature hot plate chamber at a temperature of 400° C. for 1 minute to 2 minutes to form each of the resist underlayer films.

Separately from the above, an amorphous carbon layer (hereinafter, referred to as “ACL”) having a thickness of 0.4 μm was deposited on a silicon wafer using chemical vapor deposition (CVD). Then, an etching process was performed for 60 seconds by using a CF₄ plasma gas.

Each of the above-formed resist underlayer films was dry-etched for 60 seconds by using the CF₄ plasma gas. Then, the etched thin film thickness (Å) per second (sec) was measured. The results thereof are specified in Table 9 below. In addition, the thickness ratio (%) of the resist underlayer film remaining after the dry etching compared to the initial thickness thereof was measured. The results thereof are specified in Table 9 below.

TABLE 9

| | Etched thin film | |
|---|---|---|
| | thickness per second | Thickness ratio |
| Sample | (Å/s) | after etching (%) |
| Example 1 | 19.8 | 86.0 |
| Example 2 | 19.6 | 86.9 |
| Example 3 | 19.7 | 86.2 |
| Example 4 | 19.5 | 87.1 |
| Example 5 | 23.5 | 72.3 |
| Example 6 | 19.0 | 89.4 |
| Example 7 | 20.7 | 82.0 |
| Example 8 | 21.7 | 78.5 |
| Example 9 | 20.3 | 83.7 |
| Example 10 | 19.7 | 86.3 |
| Example 11 | 19.5 | 87.2 |
| Example 12 | 20.3 | 84.0 |
| Example 13 | 20.0 | 85.0 |
| Example 14 | 19.7 | 86.4 |
| Example 15 | 20.2 | 84.3 |
| Example 16 | 20.9 | 81.3 |
| Reference | 23.5 | 72.3 |
| Example 1 | | |
| Reference | 23.5 | 72.3 |
| Example 2 | | |
| Reference | — | 100.0 |
| Example 3 | | |
| (ACL) | | |
| Comparative | 24.5 | 69.5 |
| Example 1 | | |
| Comparative | 24.3 | 70.0 |
| Example 2 | | |

Referring to Table 9, in Examples 1 to 16, it was confirmed that the etched thickness per second (sec) by the CF₄ plasma gas was significantly reduced compared to that in Comparative Examples 1 and 2.

Particularly, in Examples 1 to 4, 6, 10, 11, and 14, it was confirmed that the thickness ratio of the resist underlayer film remaining after the etching, compared to the thickness ratio of the ACL, which was 100%, was all 86% or more, which was higher than that in Comparative Examples 1 and 2.

Analyzing this together, since the resist underlayer films prepared according to Examples 1 to 4, 6, 10, 11, and 14 had rather high etching resistance against the CF₄ plasma gas, it is inferred that a hardmask film having a relatively small thickness can be formed. In addition, it is inferred that the occurrence rate of the bending of a pattern can be significantly reduced during the etching by forming a uniform film layer compared to that in Comparative Examples 1 and 2.

Comparing Examples 1 to 4, when an amine group or a hydroxyl group was included as the repeating unit, it was confirmed that a similar level of the etching resistance characteristics against the CF₄ plasma gas were exhibited.

Comparing Example 1 with Examples 7 and 8, when a structure A in Formula 1 did not contain a polar functional group, it was confirmed that the etching resistance characteristics against the CF₄ plasma gas were further enhanced.

Comparing Examples 1 and 9 with Examples 14 and 15, when fluorenone was used as a compound capable of terminating polymer chain extension, it was confirmed that the etching resistance characteristics against the CF₄ plasma gas were further enhanced, compared to when cyclohexanone was used.

Comparing Examples 1 and 12 and Examples 2 and 13 to each other, when the structure A in Formula 1 did not contain a polar functional group, it was confirmed that the etching resistance characteristics against the CF₄ plasma gas were further enhanced.

Comparing Examples 1 and 16, as R₁ in Formula 1 did not form a fused ring with a benzene ring, it was confirmed that the etching resistance characteristics against the CF₄ plasma gas were further enhanced.

Comparing Example 5 and Reference Examples 1 and 2, when the compound had a weight average molecular weight in a range of 1,000 g/mol to 10,000 g/mol, it was inferred that etching resistance characteristics against the CF₄ plasma gas were able to be further enhanced.

Considering the above experimental results comprehensively, when using the compound prepared according to an embodiment of the present disclosure, the multiple structures of the thermal cross-linking functional groups cause a thermal cross-linking action, thereby increasing the degree of cross-linking. In addition, cross-linking at a low temperature can be facilitated, thereby increasing the density of the resist underlayer film. Furthermore, as the density of such film layer increases, etching resistance can be enhanced during the etching by using the CF₄ plasma gas. Moreover, the resist underlayer film having an even smaller thickness can be formed. As a result, it is inferred that the occurrence rate of the bending of a pattern can be reduced during the etching.

### Experimental Example 1-2: Evaluation of Etching Resistance

1) Average Etched Thin Film Thickness Per Second

The resist underlayer films (having a thickness of 0.6 μm), newly formed in the same manner as in Experimental Example 1, were each independently dry-etched for 30 seconds (at an etching rate of 10%), 100 seconds (at an etching rate of 30%), and 200 seconds (at an etching rate of 60%) by using an O₂/N₂ plasma mixture gas. Then, the average etched thin film thickness (Å) per second (sec) was measured. The results thereof are specified in Table 10 below.

An ACL having a thickness of 0.5 μm was deposited on a silicon wafer using chemical vapor deposition. Then, etching processes were each independently performed for 30 seconds (at an etching rate of 10%), 100 seconds (at an etching rate of 30%), and 200 seconds (at an etching rate of 60%) by using the O₂/N₂ plasma mixture gas.

2) Average Ratio of Etched Thickness

The thickness ratio (%) of each of the resist underlayer films remaining after the above dry etching compared to the initial thickness was measured. The results thereof are specified in Table 9 below. The thickness ratio (%) of each of the resist underlayer films remaining after the dry etching was converted based on the thickness of the ACL, which was 100%. In addition, bulk etch rate (BER) was calculated by substituting the etched thin film thickness (Å), with respect to each etching time, into Equation 1 below. Furthermore, standard deviation values (hereinafter referred to as “STDEV”) thereof are specified in Table 10 below.

Bulk etch rate (BER)=(initial thickness of thin film−thickness of thin film remaining after etching)/etching time [Equation 1]

TABLE 10

| | Average etched thin | Average ratio of | |
|---|---|---|---|
| | film thickness per | etched thickness | O₂/N₂ bulk etch |
| Sample | second (Å/s) | (%) | rate (STDEV) |
| Example 1 | 17.7 | 78.7 | 0.08 |
| Example 2 | 17.6 | 79.2 | 0.07 |
| Example 3 | 18.1 | 76.7 | 0.16 |
| Example 4 | 18.0 | 77.2 | 0.18 |
| Example 5 | 21.6 | 64.4 | 0.30 |
| Example 6 | 17.1 | 81.2 | 0.06 |
| Example 7 | 18.9 | 73.4 | 0.14 |
| Example 8 | 19.9 | 70.0 | 0.12 |
| Example 9 | 18.1 | 76.8 | 0.21 |
| Example 10 | 17.4 | 79.9 | 0.10 |
| Example 11 | 17.1 | 81.3 | 0.11 |
| Example 12 | 17.8 | 78.3 | 0.13 |
| Example 13 | 17.6 | 79.0 | 0.12 |
| Example 14 | 17.7 | 78.6 | 0.10 |
| Example 15 | 18.2 | 76.5 | 0.22 |
| Example 16 | 18.9 | 73.5 | 0.14 |
| Reference | 21.6 | 64.4 | 0.30 |
| Example 1 | | | |
| Reference | 21.6 | 64.4 | 0.30 |
| Example 2 | | | |
| Reference | — | 100.0 | — |
| Example 4 | | | |
| (ACL) | | | |
| Comparative | 22.5 | 61.9 | 0.38 |
| Example 1 | | | |
| Comparative | 22.8 | 61.0 | 0.45 |
| Example 2 | | | |

Referring to Table 10, in Examples 1 to 16, the average etched thin film thickness per second (sec) by the O₂/N₂ plasma mixture gas was 21.6 Å/s or less, which was lower than that in Comparative Examples 1 and 2. As a result, it was confirmed that etching resistance was significantly enhanced.

Particularly, in all of Examples 1 to 4, 6, and 9 to 16, the thickness ratio of the resist underlayer film remaining after the etching was 76% or more, compared to the thickness of the ACL, which was 100%. As a result, it was confirmed that the etching resistance which was higher than that in Comparative Examples 1 and 2 was realized.

Particularly, in Examples 1, 2, 6, 10, and 14, the STDEV value of the BER for the O₂/N₂ plasma mixture gas was significantly lower than that in Comparative Examples 1 and 2. As a result, it is inferred that the further uniform etching rate for the O₂/N₂ plasma mixture gas can be obtained.

Comparing Examples 1 to 4, when a hydroxyl group was contained as the repeating unit instead of an amine group, the average etched thickness per second decreased and the average ratio of etched thickness increased. In addition, the BER for the O₂/N₂ plasma mixture gas decreased. As a result, it was confirmed that the overall etching resistance characteristics were further enhanced.

Comparing Example 1 with Examples 7 and 8, when a structure A in Formula 1 did not contain a polar functional group, it was confirmed that the etching resistance characteristics against the O₂/N₂ plasma mixture gas were further enhanced.

Comparing Examples 1 and 9 with Examples 14 and 15, when fluorenone was used as a compound capable of terminating polymer chain extension, it was confirmed that the etching resistance characteristics against the O₂/N₂ plasma mixture gas were further enhanced, compared to when cyclohexanone was used.

Comparing Examples 1 and 12 and Examples 2 and 13 to each other, when the structure A in Formula 1 did not contain a polar functional group, it was confirmed that the etching resistance characteristics against the O₂/N₂ plasma mixture gas were further enhanced.

Comparing Examples 1 and 16, as R₁ in Formula 1 did not form a fused ring with a benzene ring, it was confirmed that the etching resistance characteristics against the O₂/N₂ plasma mixture gas were further enhanced.

Comparing Example 5 and Reference Examples 1 and 2, when the compound had a weight average molecular weight in a range of 1,000 g/mol to 10,000 g/mol, it is inferred that etching resistance characteristics against the O₂/N₂ plasma mixture gas can be further enhanced.

Considering the above experimental results comprehensively, when the compound prepared according to an embodiment of the present disclosure is used, the multiple structures of the thermal cross-linking functional groups cause a thermal cross-linking action, thereby increasing the degree of cross-linking. In addition, cross-linking at a low temperature can be facilitated, thereby increasing the density of the resist underlayer film. Furthermore, as the density of such film layer increases, etching resistance can be enhanced during the etching by using the O₂/N₂ plasma mixture gas. Moreover, the resist underlayer film having an even smaller thickness can be formed. As a result, it is inferred that the occurrence rate of the bending of a pattern can be reduced during the etching.

### Experimental Example 2: Evaluation of Gap-Filling Characteristics

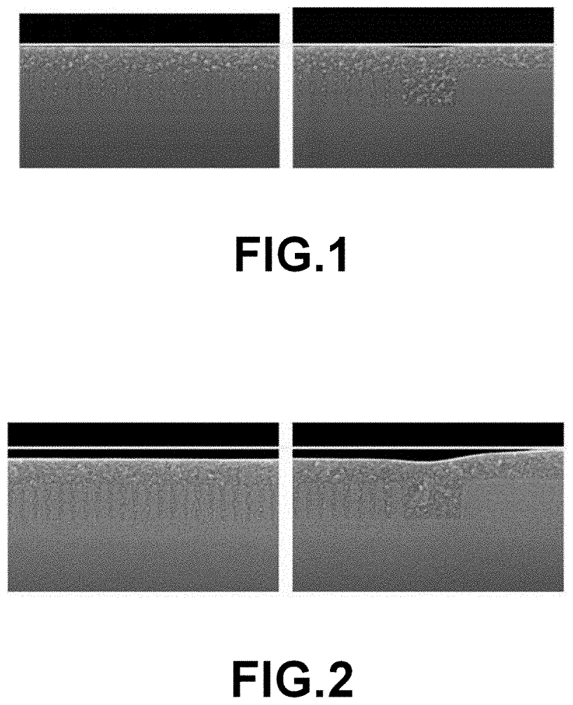

The resist underlayer film compositions prepared according to Preparation Example 1 were each independently applied on a silicon wafer on which line-and-space pattern with a line width of 15 nm and a pattern height of 130 nm was formed, by spin coating in a thickness of 130 nm. The coated resultant was baked in a high-temperature hot plate chamber at a temperature of 400° C. for 1 minute to 2 minutes to form each of the resist underlayer films. A cross-section of such formed resist underlayer film was observed using a field emission scanning electron microscope (FE-SEM, S-4300 manufactured by Hitachi Corporation), and the results are shown in Table 11 below and FIGS. 1 to 6.

When an aspect ratio of the resist underlayer film formed on a part having a large pattern thickness (a line part) and a part having a small pattern thickness (a space part) was 3 nm or less, “extremely good” was marked. When the aspect ratio was higher than 3 nm and 10 nm or less, “good” was marked. When the aspect ratio was higher than 10 nm and 15 nm or less, “poor” was marked. When the aspect ratio was higher than 15 nm, “extremely poor” was marked.

In addition, when voids were generated in such formed underlayer film, “0” was marked. When there were no voids, “X” was marked.

TABLE 11

| | | Presence of voids |
|---|---|---|
| Sample | Aspect ratio performance | (◯/X) |
| Example 1 | Extremely good | X |
| Example 2 | Extremely good | X |
| Example 3 | Good | X |
| Example 4 | Good | X |
| Example 5 | Good | X |
| Example 6 | Good | X |
| Example 7 | Good | X |
| Example 8 | Good | X |
| Example 9 | Good | X |
| Example 10 | Extremely good | X |
| Example 11 | Extremely good | X |
| Example 12 | Good | X |
| Example 13 | Good | X |
| Example 14 | Extremely good | X |
| Example 15 | Good | X |
| Example 16 | Good | X |
| Reference | Poor | X |
| Example 1 | | |
| Reference | Extremely Poor | ◯ |
| Example 2 | | |
| Comparative | Extremely Poor | X |
| Example 1 | | |
| Comparative | Poor | ◯ |
| Example 2 | | |

Comparing Examples 1 to 4, since a hydroxyl group was contained as the repeating unit instead of an amine group, the aspect ratio of the resist underlayer film formed on the part having the large pattern thickness (the line part) and the part having the small pattern thickness (the space part) further decreased. As a result, it was confirmed that aspect ratio performance was better.

A ratio of a total number of polar functional groups to a total number of rings (the total number of the polar functional groups: the total number of the rings) in Example 5 was about 1:0.4, and a ratio of a total number of polar functional groups to a total number of rings (the total number of the polar functional groups: the total number of the rings) in Example 6 was about 1:4.2. On the other hand, in Examples 1, 2, 10, 11, and 14, each ratio of a total number of polar functional groups to a total number of rings (the total number of the polar functional groups: the total number of the rings) specifically fell within the range of 1:1 to 4, so the aspect ratio of the resist underlayer film formed on the part having the large pattern thickness (the line part) and the part having the small pattern thickness further decreased (the space part). As a result, it was confirmed that the aspect ratio performance was better.

Comparing Example 1 with Examples 7 and 8, when a structure A in Formula 1 did not contain a polar functional group, the aspect ratio of the resist underlayer film formed on the part having the large pattern thickness (the line part) and the part having the small pattern thickness (the space part) further decreased. As a result, it was confirmed that aspect ratio performance was better.

Comparing Examples 1 and 9 with Examples 14 and 15, when fluorenone was used as a compound capable of terminating polymer chain extension, the aspect ratio of the resist underlayer film formed on the part having the large pattern thickness (the line part) and the part having the small pattern thickness (the space part) further decreased, compared to when cyclohexanone was used. As a result, it was confirmed that the aspect ratio performance was better.

Comparing Examples 1 and 12 and Examples 2 and 13 to each other, when the structure A in Formula 1 did not contain a polar functional group, the aspect ratio of the resist underlayer film formed on the part having the large pattern thickness (the line part) and the part having the small pattern thickness (the space part) further decreased. As a result, it was confirmed that the aspect ratio performance was better.

Comparing Examples 1 and 16, as R₁ in Formula 1 did not form a fused ring with a benzene ring, the aspect ratio of the resist underlayer film formed on the part having the large pattern thickness (the line part) and the part having the small pattern thickness (the space part) further decreased. As a result, it was confirmed that the aspect ratio performance was better.

In addition, unlike in Comparative Examples or Reference Examples, no voids were generated in any of Examples. As a result, it was confirmed that gap-filling characteristics were good.

Comparing Example 5 with Reference Examples 1 and 2, when the compound had a weight average molecular weight in the range of 1,000 g/mol to 10,000 g/mol, the aspect ratio of the resist underlayer film formed on the part having the large pattern thickness (the line part) and the part having the small pattern thickness (the space part) was extremely low. As a result, it was confirmed that the aspect ratio performance was better.

FIG. 1 shows FE-SEM images of the resist underlayer film prepared according to Example 1.

Referring to FIG. 1, it was confirmed that the gap-filling characteristics and the aspect ratio of the resist underlayer film prepared according to Example 1 were extremely low. The compound, prepared according to Example 1, did not form a large polymer structure, but formed multiple structures of the thermal cross-linking functional groups. As a result, when a thermal cross-linking action was caused, the degree of cross-linking increased. In addition, cross-linking at a low temperature was facilitated, so the density of the resist underlayer film increased due to the compound prepared according to Example 1, thereby preventing thermal shrinkage. As a result, it was confirmed that the aspect ratio became low.

FIG. 2 shows FE-SEM images of the resist underlayer prepared according to Comparative Example 1.

Referring to FIG. 2, the gap-filling characteristics could be obtained by adjusting the molecular weight of the compound prepared according to Comparative Example 1. However, it was confirmed that the thickness shrinkage rate of the underlayer film increased due to the low degree of cross-linking, thereby increasing the aspect ratio.

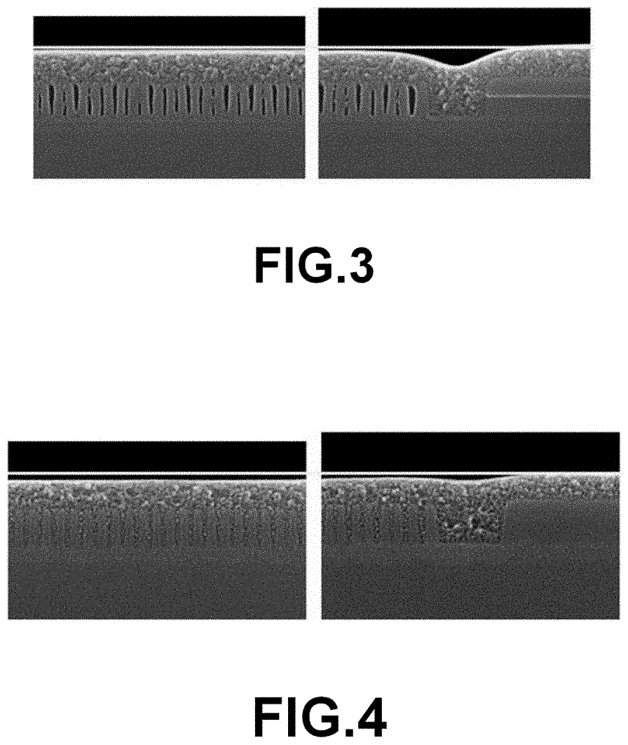

FIG. 3 shows FE-SEM images of the resist underlayer prepared according to Comparative Example 2.

Referring to FIG. 3, the compound prepared according to Comparative Example 2 formed a large polymer structure, compared to the compound prepared according to Comparative Example 1. As a result, the thickness shrinkage rate of the resist underlayer film prepared according to Comparative Example 2 was relatively lower than that of the resist underlayer film prepared according to Comparative Example 1. However, it was confirmed that the gap-filling characteristics were extremely poor, so voids were generated.

FIG. 4 shows FE-SEM images of the resist underlayer prepared according to Comparative Example 3.

Referring to FIG. 4, the compound prepared according to Comparative Example 3 had a low molecular structure and a high degree of cross-linking, which had an effect of reducing the aspect ratio in a pattern having a small critical dimension (CD), which was the distance between the patterns. However, the thickness shrinkage rate increased when the underlayer film had a relatively large CD. As a result, it was confirmed that the aspect ratio increased compared to that in the Example 1.

Referring to Table 11, the resist underlayer film prepared according to the remaining Examples, other than Example 1, had good gap-filling characteristics. As a result, it is inferred that even when performing a gap-filling process on a micro pattern having a small CD, the aspect ratio may be low, and voids may be prevented from being generated.

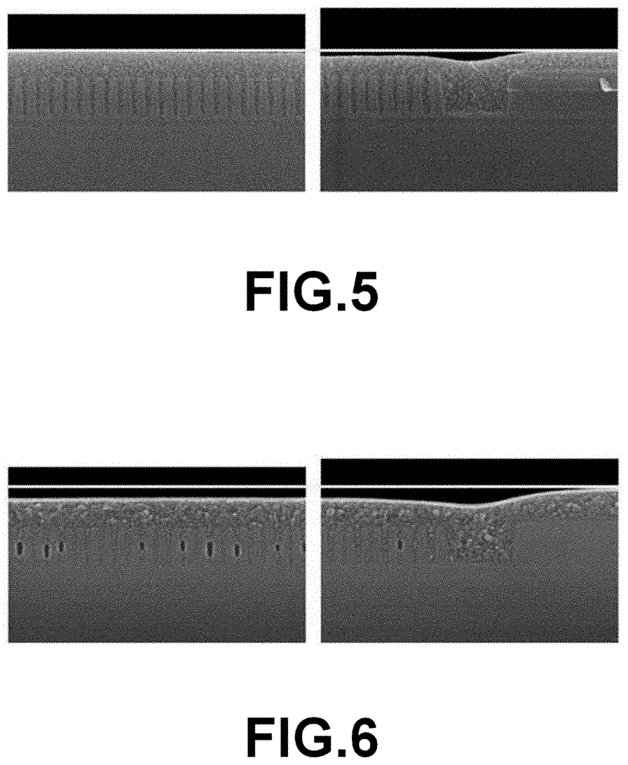

FIG. 5 shows FE-SEM images of the resist underlayer prepared according to Reference Example 2.

Referring to FIG. 5, the resist underlayer film prepared according to Reference Example 1 was made of the compound having the weight average molecular weight of less than 1,000 g/mol. Even though the voids were not generated, it was confirmed that planarization characteristics were deteriorated and dishing was exhibited.

FIG. 6 shows FE-SEM images of the resist underlayer prepared according to Reference Example 2.

Referring to FIG. 6, the resist underlayer film prepared according to Reference Example 2 was made of the compound having the weight average molecular weight of more than 10,000 g/mol. Since the gap-filling characteristics were extremely deteriorated, voids were generated. In addition, it was confirmed that planarization characteristics were extremely deteriorated.

Comparing the experimental results of FIGS. 5 and 6 with Example 5, when the weight average molecular weight of the compound fell within the range of 1,000 g/mol to 10,000 g/mol, the density of the resist underlayer film increased to enhance etching resistance. In addition, the pattern bending during an etching process was able to be effectively prevented. Furthermore, when the resist underlayer film composition was used to coat a substrate, the gap-filling characteristics were improved. As a result, it was confirmed that the occurrence of voids was able to be prevented and a planar underlayer film was able to be implemented.

While the present disclosure has been described with reference to exemplary embodiments, it is to be understood that the scope of the present disclosure is not limited to the disclosed exemplary embodiments. Modified forms are also included within the scope of the present disclosure.

What is claimed is:

1. A compound comprising a repeating unit represented by Formula 1, 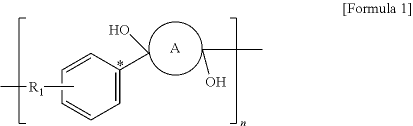
wherein in Formula 1,
R₁ has 1 to 5 substituents that are present in a carbon (*)-containing benzene ring by substitution,
A is a substituted or unsubstituted monocyclic ring or polycyclic ring, and
n is a number of the repeating units, wherein n is in a range of 1 to 55.

2. The compound of claim 1, wherein the monocyclic ring or polycyclic ring comprises a polar functional group, and
wherein the polar functional group comprises at least one selected from the group consisting of a hydroxyl group, an amine group, a thiol group, and a carboxyl group.

3. The compound of claim 1, wherein R₁ has 2 or 3 substituents which are meta-substituted and para-substituted around the carbon (*), and
wherein the 2 or 3 substituents are bonded to each other to form a monocyclic ring or polycyclic ring.

4. The compound of claim 1, wherein R₁ is a molecular structure represented by Formula 2, 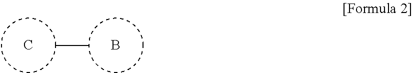
wherein in Formula 2,
B is bonded to the carbon (*)-containing benzene ring in Formula 1 and comprises a molecular structure of a monocyclic aromatic hydrocarbon or polycyclic aromatic hydrocarbon which is substituted with a polar functional group, and
C comprises a molecular structure of a monocyclic aromatic hydrocarbon or polycyclic aromatic hydrocarbon which is substituted with a polar functional group.

5. The compound of claim 4, wherein the polar functional groups of B and C are each independently at least one selected from the group consisting of a hydroxyl group, an amine group, a thiol group, and a carboxyl group.

6. The compound of claim 1, wherein the compound comprises first and second molecular structures at first and second terminal ends, respectively, and
wherein the first and second terminal ends are same or different.

7. The compound of claim 6, wherein the first and second molecular structures are each independently a molecular structure derived from a ketone-based compound or a molecular structure derived from an aryl halide-based compound, and
wherein the first and second molecular structures each independently comprise a polar functional group.

8. The compound of claim 7, wherein the ketone-based compound is a fluorenone-based compound.

9. The compound of claim 1, wherein a ratio of a total number of polar functional groups to a total number of rings (the total number of the polar functional groups: the total number of the rings) in the compound is in a range of about 1:1 to about 1:4.

10. The compound of claim 1, wherein the compound has a weight average molecular weight in a range of about 800 g/mol to about 20,000 g/mol.

11. The compound of claim 10, wherein the compound has a weight average molecular weight in a range of about 1,000 g/mol to about 10,000 g/mol.

12. A resist underlayer film composition comprising the compound of claim 1 and a solvent.

13. The resist underlayer film composition of claim 12, wherein the compound included in the resist underlayer film composition accounts for about 2.0% to about 30.0% by weight with respect to a total weight of the resist underlayer film composition.

14. The resist underlayer film composition of claim 12, further comprising an additive.

15. The resist underlayer film composition of claim 14, wherein the additive comprises a cross-linking agent, an acid catalyst, an acid generator, a surfactant, or any combinations thereof.

16. A resist underlayer film comprising a cured product of the resist underlayer film composition of claim 12.

17. The resist underlayer film of claim 16, wherein the resist underlayer film has a thickness in a range of about 0.03 μm to about 1.50 μm.
